(19) United States
(12) Patent Application Publication
(10) Pub. No.: US 2024/0075102 A1
(43) Pub. Date: Mar. 7, 2024

(54) Macrophage Stimulating 1 Receptor (MST1R) Variants And Uses Thereof

(71) Applicant: Regeneron Pharmaceuticals, Inc., Tarrytown, NY (US)

(72) Inventors: Julie Horowitz, Tarrytown, NY (US); Aris Baras, Tarrytown, NY (US); Manuel Allen Revez Ferreira, Tarrytown, NY (US)

(21) Appl. No.: 18/355,108

(22) Filed: Jul. 19, 2023

Related U.S. Application Data
(63) Continuation of application No. 16/990,744, filed on Aug. 11, 2020, now Pat. No. 11,752,197.
(60) Provisional application No. 62/885,402, filed on Aug. 12, 2019.

(51) Int. Cl. A61K 38/18; A61K 31/12; A61K 31/4741; A61K 38/17; A61K 38/48; A61P 1/00; C07K 16/22; C07K 16/28; C12Q 1/6883; G01N 33/68

(52) U.S. Cl. CPC A61K 38/18; A61K 31/12; A61K 31/4741; A61K 38/1709; A61K 38/177; A61K 38/1796; A61K 38/482; A61K 38/4846; A61K 38/4853; A61P 1/00; C07K 16/22; C07K 16/2863; C12Q 1/6883; G01N 33/6893; C12Q 2600/118; C12Q 2600/156; G01N 33/48; G01N 2333/475; G01N 2333/71; G01N 2800/065

(57) ABSTRACT

Methods of treating patients having inflammatory bowel disease (IBD) or primary sclerosing cholangitis (PSC) are provided herein.

### REFERENCE TO A SEQUENCE LISTING

This application includes a Sequence Listing submitted electronically as a text file named 189238029015EQ, created on Aug. 8, 2020, with a size of 201 kilobytes. The Sequence Listing is incorporated herein by reference.

### FIELD

The present disclosure provides methods of treating patients having inflammatory bowel disease (IBD) or primary sclerosing cholangitis (PSC).

### BACKGROUND

IBD is group of inflammatory conditions of the colon and small intestine that cause over 50,000 deaths annually. The causes of IBD are complex, and contributing factors may include diet, genetics, and the composition of an individual's gut microflora. Medical treatment is largely based on factors specific to an individual.

Crohn's disease (CD) and ulcerative colitis (UC) are among the most common forms of IBD. Both CD and UC are inflammatory diseases, but while UC is localized to the colon, CD can affect any part of the gastrointestinal tract, from mouth to anus. Neither CD nor UC are currently medically curable, and current treatments range from surgical removal of parts of the intestine to administration of anti-inflammatory and/or immunosuppressive drugs. Unfortunately, current treatments for CD and UC are often ineffective and can result in significant side effects.

PSC was originally defined as a chronic cholestatic liver disease characterized by fibrosing inflammation of segments of the extrahepatic bile ducts. PSC results in a progressive narrowing or obliteration of bile duct lumens, progression to secondary biliary cirrhosis, with complications of portal hypertension, hepatic failure, and cholangiocarcinoma. It is an idiopathic disorder characterized by inflammation and obliteration of both intra-hepatic and extrahepatic bile ducts. Further, roughly two-thirds or more of all PSC patients have concomitant IBD. However, the relationship between these two diseases remains undefined. Cholangiocytes account for 3%-5% of the hepatic cell population and line a complex network of interconnecting conduits in the liver, termed the intrahepatic biliary ductal system. One of the pathological conditions manifested in both the intrahepatic and extrahepatic bile ducts is PSC. Medical treatment of PSC has included corticosteroids, antibiotics, immunosuppressants, and cholecystogues alone or in combination. In general, results with all have been disappointing, and, in the absence of a liver transplant, the median age from diagnosis to death is 10 years. Thus, there is a continuing need for new methods and compositions for the treatment of IBD and/or PSC.

MST1 encodes macrophage stimulating protein (MSP; alternately designated MST1 protein), a liver-secreted protein that binds to its cognate receptor MST1R (alternately designated Recepteur d'Origine Nantais, or RON), which is expressed on macrophages, Kupffer cells, and epithelial cells of the intestine, among other cell types. MST1/MST1R signaling drives macrophage polarization, cellular chemotaxis, and epithelial wound repair. Previous genome-wide association studies (GWAS) in PSC and IBD patients and healthy controls have identified a common missense variant in MST1 (3:49684099:G:A; r53197999) that associates with increased risk of PSC (OR 1.3, p=2e-26) and IBD (OR 1.2, p=1e-47) in published meta-GWAS. Functional studies have shown that 3:49684099:G:A rs3197999 associates with decreased MST1 protein levels in human serum. Collectively, these data indicate that decreased MST1 is associated with increased risk of PSC and IBD.

MST1R is a cell surface receptor for MST1 with tyrosine kinase activity. The mature form of this protein is a heterodimer of disulfide-linked alpha and beta subunits, generated by proteolytic cleavage of a single-chain precursor. MST1R functions as a tyrosine kinase that transduces signals from the extracellular matrix into the cytoplasm by binding to MST1 ligand. To date, MST1R has not been independently associated with IBD or PSC in genetic association studies.

### SUMMARY

The present disclosure provides methods of treating a patient having IBD, the method comprising administering to the patient an agonist of the MST1/MST1R pathway.

The present disclosure also provides methods of treating a patient having PSC, the method comprising administering to the patient an agonist of the MST1/MST1R pathway.

The present disclosure also provides, in some embodiments, such methods wherein the presence or absence of an MST1 and/or MST1R variant nucleic acid molecule or variant polypeptide associated with an increased risk of developing IBD and/or PSC is detected in a biological sample from the patient.

### BRIEF DESCRIPTION OF THE FIGURES

The accompanying figures, which are incorporated in and constitute a part of this specification, illustrate several aspects and together with the description serve to explain some principles of the present disclosure.

The patent or application file contains at least one drawing executed in color. Copies of this patent or patent application publication with color drawings will be provided by the Office upon request and payment of the necessary fee.

FIG. 1 shows MST1R protein expression in mouse peritoneal macrophages, liver Kuppfer cells, bone marrow macrophages, colon lamina propria macrophages, and colon epithelial cells by flow cytometry (Kauder et al., PLoS ONE, 2013, 8, e83958).

FIG. 2 shows MST1R protein domains highlighting loss-of-function and predicted-deleterious missenses associated with IBD and found in Table 1.

FIG. 3 shows results of meta-analysis of association of MST1 Arg651STOP pLoF mutant with PSC (panel A) and IBD (panel B).

FIG. 4 shows MST1 RNA expression (panel A) and MST1R RNA (panel B) in control and DSS-induced model of colitis mice.

FIG. 5 shows MST1 expression in serum of control and IBD human patients.

FIG. 6 shows MST1 expression (panel A) and MST1R expression (panel B) in intestinal epithelial cells obtained from healthy controls and human patients having UC or a functional gastrointestinal disorder (FGID).

FIG. 7 shows results from single cell RNA sequencing in an IBD population (ulcerative colitis, Crohn's disease and functional gastrointestinal disorders) showing MST1R expression in epithelial cells (panel A) and intestinal immune cells (panel B) and MST1 expression in epithelial cells (panel C) and intestinal immune cells (panel D).

FIG. 8 shows consistent MST1-HDD expression in the livers of 3 control-HDD and 3 mMst1-HDD mice.

FIG. 9 shows serum levels of WT and variant MST1 and MST1R in patients with IBD.

FIG. 10 shows meta analysis of MST1R predicted loss of function with gene burden associations to increased odds of IBD and PSC.

FIG. 11 shows expression of a truncated MST1R protein resulting from the G116fs mutation.

FIG. 12 shows that the MST1R G116fs mutant protein is not expressed in HEK cells and does not activate its downstream targets.

FIG. 13 shows a schematic representation of MST1 constructs used in transfection experiments.

FIG. 14 shows expression and secretion of variant MST1 proteins.

FIG. 15 shows body weight changes in mice expressing the C672A MDT1 mutant protein.

FIG. 16 shows analysis of plasma levels of the C672A MDT1 mutant protein.

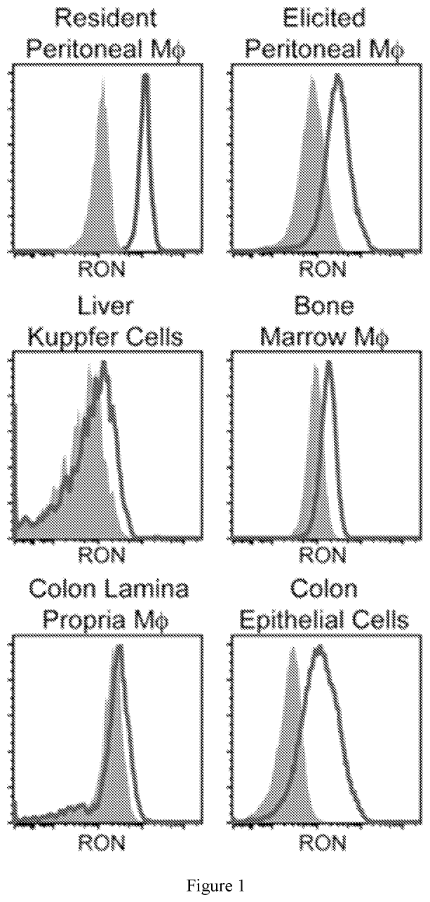

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

As used herein, a “nucleic acid,” a “nucleic acid molecule,” a “nucleic acid sequence,” a “polynucleotide,” or an “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “comprising” may be replaced with “consisting” or “consisting essentially of” in particular embodiments as desired.

As used herein, the phrase “corresponding to”, or grammatical variations thereof, when used in the context of the numbering of a particular amino acid or nucleotide sequence or position refers to the numbering of a specified reference sequence when the particular amino acid or nucleotide sequence is compared to the reference sequence (e.g., with the reference sequence herein being the nucleic acid molecule or polypeptide of (wild type) MST1R). In other words, the residue (e.g., amino acid or nucleotide) number or residue (e.g., amino acid or nucleotide) position of a particular polymer is designated with respect to the reference sequence rather than by the actual numerical position of the residue within the particular amino acid or nucleotide sequence. For example, a particular amino acid sequence or nucleotide sequence can be aligned to a reference sequence by introducing gaps to optimize residue matches between the two sequences. In these cases, although the gaps are present, the numbering of the residue in the particular amino acid or nucleotide sequence is made with respect to the reference sequence to which it has been aligned. A variety of computational algorithms exist that can be used for performing a sequence alignment to identify a nucleotide or amino acid position in one polymeric molecule that corresponds to a nucleotide or amino acid position in another polymeric molecule. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

It has been observed in accordance with the present disclosure that an aggregate burden of certain variations in MST1R associate with a risk of developing IBD or PSC. It is believed that variants in MST1R gene or protein have not been associated with IBD or PSC in genome-wide or exome-wide association studies. Therefore, humans having MST1R alterations that associate with IBD or PSC may be treated such that IBD or PSC is inhibited, the symptoms thereof are reduced, and/or development of symptoms is repressed. It is also believed that humans having IBD or PSC may be treated with molecules that promote MST1/MST1R signaling.

For purposes of the present disclosure, any particular human can be categorized as having any combination of the following MST1 and/or MST1R genotypes: i) MST1 and/or MST1R reference; ii) heterozygous for an MST1 and/or MST1R variant nucleic acid molecule associated with an increased risk of developing IBD and/or PSC, and iii) homozygous for an MST1 and/or MST1R variant nucleic acid molecule associated with an increased risk of developing IBD and/or PSC. A human is MST1 and/or MST1R reference when the human does not have a copy of an MST1 and/or MST1R variant nucleic acid molecule associated with an increased risk of developing IBD and/or PSC. A human is heterozygous for an MST1 and/or MST1R variant nucleic acid molecule associated with an increased risk of developing IBD and/or PSC when the human has a single copy of an MST1 and/or MST1R variant nucleic acid molecule associated with an increased risk of developing IBD and/or PSC. A human is homozygous for an MST1 and/or MST1R variant nucleic acid molecule associated with an increased risk of developing IBD and/or PSC when the human has two copies of any of the MST1 and/or MST1R variant nucleic acid molecule associated with an increased risk of developing IBD and/or PSC.

An MST1 and/or MST1R variant nucleic acid molecule associated with an increased risk of developing IBD and/or PSC is any MST1 and/or MST1R nucleic acid molecule (such as, a genomic nucleic acid molecule, an mRNA molecule, or a cDNA molecule produced from the mRNA molecule) encoding an MST1 and/or MST1R polypeptide having a partial loss-of-function, a complete loss-of-function, a predicted partial loss-of-function, or a predicted complete loss-of-function (collectively, “predicted loss-of-function” variant nucleic acid molecule). A human who has an MST1 and/or MST1R polypeptide having a partial loss-of-function (or predicted partial loss-of-function) is hypomorphic for MST1 and/or MST1R. The MST1 and/or MST1R predicted loss-of-function variant nucleic acid molecule can be any one or more of the variant nucleic acid molecules described herein.

An MST1 and/or MST1R variant nucleic acid molecule associated with an increased risk of developing IBD and/or PSC is also any MST1 and/or MST1R deleterious missense variant nucleic acid molecule. The MST1 and/or MST1R deleterious missense variant nucleic acid molecule can be any one or more of the variant nucleic acid molecules described herein.

For human subjects or patients that are genotyped or determined to be heterozygous or homozygous for an MST1 and/or MST1R variant nucleic acid molecule associated with an increased risk of developing IBD and/or PSC, such human subjects or patients are associated with having increased odds of developing IBD and/or PSC.

In any of the embodiments described herein, the MST1 and/or MST1R variant nucleic acid molecule associated with an increased risk of developing IBD and/or PSC can be any MST1 and/or MST1R nucleic acid molecule (such as, for example, genomic nucleic acid molecule, mRNA molecule, or cDNA molecule produced from the mRNA molecule) encoding an MST1 and/or MST1R polypeptide having a partial loss-of-function, a complete loss-of-function, a predicted partial loss-of-function, or a predicted complete loss-of-function. In any of the embodiments described herein, the MST1 and/or MST1R variant nucleic acid molecule associated with an increased risk of developing IBD and/or PSC can also be any MST1 and/or MST1R missense variant nucleic acid molecule (such as, for example, genomic nucleic acid molecule, mRNA molecule, or cDNA molecule produced from the mRNA molecule).

The nucleotide sequence of an MST1R reference genomic nucleic acid molecule is set forth in SEQ ID NO:1, which is 16,636 nucleotides in length. The first nucleotide recited in SEQ ID NO:1 corresponds to the nucleotide at position 49,903,637 of chromosome 3 (see, hg38_knownGene_ENSG00000164078.12).

Numerous variant genomic nucleic acid molecules of MST1R exist, including, but not limited to (using the human genome reference build GRch38): 3:49903264:CG:C, 3:49903084:C:T, 3:49890026:G:T, 3:49902417:A:G, 3:49902560:A:T, and 3:49903387:G:T. Thus, for example, using the reference genomic nucleotide sequence (SEQ ID NO:1) as a base sequence (with the first nucleotide listed therein designated as position 49,903,637), the first listed variant (3:49903264:CG:C) has a CG dinucleotide replaced with a cytosine (designated the “variant nucleotide”) at position 49,903,264, effectively deleting the guanine (designated the “variant position”). In another example, the second listed variant (3:49903084:C:T) has a cytosine replaced with a thymine (designated the “variant nucleotide”) at position 49,903,084 (designated the “variant position”). Any of these MST1R predicted loss-of-function variant genomic nucleic acid molecules can be detected in any of the methods described herein.

The nucleotide sequences of MST1R reference mRNA molecules produced through alternative splicing are set forth in SEQ ID NOs:2-10. The variant nucleotides at their respective variant positions for the variant genomic nucleic acid molecules described herein also have corresponding variant nucleotides at their respective variant positions for the variant mRNA molecules based upon the MST1R reference mRNA sequences according to SEQ ID NOs:2-10. Any of these MST1R variant mRNA molecules can be detected in any of the methods described herein.

The nucleotide sequences of MST1R reference cDNA molecules are set forth in SEQ ID NOs:11-19. The variant nucleotides at their respective variant positions for the variant genomic nucleic acid molecules described herein also have corresponding variant nucleotides at their respective variant positions for the variant cDNA molecules based upon the MST1R reference cDNA sequence according to SEQ ID NOs:11-19. Any of these MST1R variant cDNA molecules can be detected in any of the methods described herein.

The amino acid sequences of MST1R reference polypeptide isoforms are set forth in SEQ ID NOs:20-28. Using the translated nucleotide sequence of either the MST1R mRNA or cDNA molecules, the MST1R variant polypeptides have corresponding translated variant amino acids at variant positions (codons). Any of these MST1R variant polypeptides can be detected in any of the methods described herein.

The nucleotide sequence of an MST1 reference genomic nucleic acid molecule is set forth in SEQ ID NO:29, which is 5,107 nucleotides in length. The first nucleotide recited in SEQ ID NO:29 corresponds to the nucleotide at position 49,689,053 of chromosome 3 according to GRCh38/hg38 human genome assembly (see, hg38_knownGene_ENSG00000449682.2).

The nucleotide sequence of the rs3197999 variant genomic nucleic acid molecule is set forth in SEQ ID NO:30. Compared to the reference genomic nucleic acid molecule sequence (SEQ ID NO:29), the nucleotide sequence of the rs3197999 variant genomic nucleic acid molecule (SEQ ID NO:30) comprises a thymine rather than a cytosine at position 4,955. Compared to the reference genomic nucleic acid molecule sequence (SEQ ID NO:29), the nucleotide sequence of the rs3197999 variant genomic nucleic acid molecule (SEQ ID NO:30) comprises a TGC codon rather than a CGC codon at positions 4,955 to 4,957. This MST1 predicted loss-of-function variant genomic nucleic acid molecule can be detected in any of the methods described herein.

The nucleotide sequence of the rs142690032 variant genomic nucleic acid molecule is set forth in SEQ ID NO:37. Compared to the reference genomic nucleic acid molecule sequence (SEQ ID NO:29), the nucleotide sequence of the rs142690032 variant genomic nucleic acid molecule (SEQ ID NO:37) comprises a thymine rather than a cytosine at position 4,675. Compared to the reference genomic nucleic acid molecule sequence (SEQ ID NO:29), the nucleotide sequence of the rs142690032 variant genomic nucleic acid molecule (SEQ ID NO:37) comprises an ACT codon rather than an ACC codon at positions 4,673 to 4,675. This MST1 predicted loss-of-function variant genomic nucleic acid molecule can be detected in any of the methods described herein.

The nucleotide sequence of an MST1 reference mRNA molecule is set forth in SEQ ID NO:31.

The nucleotide sequence of the rs3197999 mRNA molecule is set forth in SEQ ID NO:32. Compared to the reference mRNA molecule sequence (SEQ ID NO:31), the nucleotide sequence of the rs3197999 variant mRNA molecule (SEQ ID NO:32) comprises a uracil rather than a cytosine at position 2,469. Compared to the reference mRNA molecule sequence (SEQ ID NO:31), the nucleotide sequence of the rs3197999 variant mRNA molecule (SEQ ID NO:32) comprises a UGC codon rather than a CGC codon at positions 2,469 to 2,471. This MST1 predicted loss-of-function variant mRNA molecule can be detected in any of the methods described herein.

The nucleotide sequence of the rs142690032 variant mRNA molecule is set forth in SEQ ID NO:38. Compared to the reference mRNA molecule sequence (SEQ ID NO:31), the nucleotide sequence of the rs142690032 variant mRNA molecule (SEQ ID NO:38) comprises a uracil rather than a cytosine at position 2,313. Compared to the reference mRNA molecule sequence (SEQ ID NO:31), the nucleotide sequence of the rs142690032 variant mRNA molecule (SEQ ID NO:38) comprises an ACU codon rather than an ACC codon at positions 2,311 to 2,313. This MST1 predicted loss-of-function variant genomic nucleic acid molecule can be detected in any of the methods described herein.

The nucleotide sequence of an MST1 reference cDNA molecule is set forth in SEQ ID NO:33.

The nucleotide sequence of the rs3197999 cDNA molecule is set forth in SEQ ID NO:34. Compared to the reference cDNA molecule sequence (SEQ ID NO:33), the nucleotide sequence of the rs3197999 variant cDNA molecule (SEQ ID NO:33) comprises a thymine rather than a cytosine at position 2,469. Compared to the reference cDNA molecule sequence (SEQ ID NO:33), the nucleotide sequence of the rs3197999 variant cDNA molecule (SEQ ID NO:34) comprises a TGC codon rather than a CGC codon at positions 2,469 to 2,471. This MST1 predicted loss-of-function variant cDNA molecule can be detected in any of the methods described herein.

The nucleotide sequence of the rs142690032 variant cDNA molecule is set forth in SEQ ID NO:39. Compared to the reference cDNA molecule sequence (SEQ ID NO:33), the nucleotide sequence of the rs142690032 variant cDNA molecule (SEQ ID NO:39) comprises a thymine rather than a cytosine at position 2,313. Compared to the reference cDNA molecule sequence (SEQ ID NO:33), the nucleotide sequence of the rs142690032 variant cDNA molecule (SEQ ID NO:39) comprises an ACT codon rather than an ACC codon at positions 2,311 to 2,313. This MST1 predicted loss-of-function variant cDNA molecule can be detected in any of the methods described herein.

The amino acid sequence of a reference MST1 polypeptide is set forth in SEQ ID NO:35 and is 725 amino acids long.

The amino acid sequence of the rs3197999 variant MST1 polypeptide is set forth in SEQ ID NO:36 and is 725 amino acids long. The SNP in the underlying nucleic acid molecules results in the substitution of the arginine at amino acid position 703 of the reference MST1 polypeptide (SEQ ID NO:35) with a cytosine. This rs3197999 variant MST1 polypeptide is referred to herein as Arg703Cys MST1 polypeptide.

The amino acid sequence of the rs142690032 variant MST1 polypeptide is set forth in SEQ ID NO:40 and is 650 amino acids long. The SNP in the underlying nucleic acid molecules results in a gain of a stop codon at the position corresponding to position 651 according to SEQ ID NO:35 (replacing an arginine). This results in expression of a variant MST1 polypeptide truncated at a position corresponding to position 650 according to SEQ ID NO:35. This rs142690032 variant MST1 polypeptide is referred to herein as Arg651STOP MST1 polypeptide. This variant MST1 polypeptide does not comprises the amino acids at positions corresponding to positions 651 to 725 of the reference MST1 polypeptide (SEQ ID NO:35).

In any of the embodiments described herein, the variant MST1 nucleic acid molecule or polypeptide can be or encode Arg703Cys or Arg651STOP. In any of the embodiments described herein, the variant MST1 nucleic acid molecule or polypeptide can be or encode Arg703Cys. In any of the embodiments described herein, the variant MST1 nucleic acid molecule or polypeptide can be or encode Arg651STOP.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure provides methods of treating a patient having IBD, the method comprising administering to the patient an agonist of the MST1/MST1R pathway. In any of the embodiments described herein, the IBD is ulcerative colitis (US) or Crohn's disease (CD). In any of the embodiments described herein, the CD is jejunoileitis, ileitis, ileocolitis, or Crohn's colitis, or any combination thereof. In any of the embodiments described herein, the UC is ulcerative proctitis, left-sided colitis, or extensive colitis, or any combination thereof.

Symptoms of Crohn's disease include, but are not limited to, frequent and/or recurring diarrhea, rectal bleeding, unexplained weight loss, fever, abdominal pain, cramping, fatigue, a feeling of low energy, and reduced appetite. Symptoms of ulcerative proctitis include, but are not limited to, rectal bleeding, rectal pain, and urgency in bowel movements. Symptoms of left-sided colitis include, but are not limited to, loss of appetite, weight loss, bloody diarrhea, and pain on the left side of the abdomen. Symptoms of extensive colitis include, but are not limited to, loss of appetite, bloody diarrhea, abdominal pain, and weight loss.

The present disclosure also provides methods of treating a patient having PSC, the method comprising administering to the patient an agonist of the MST1/MST1R pathway. In any of the embodiments described herein, the PSC is early stage PSC (e.g., stage 1 according to Ludwig et al., Hepatology, 1981, 1, 632-640). In any of the embodiments described herein, the PSC is late stage PSC (e.g., stage 4 according to Ludwig et al., Hepatology, 1981, 1, 632-640). In any of the embodiments described herein, the PSC is at any disease stage (e.g., stage 4 according to Ludwig et al., Hepatology, 1981, 1, 632-640). In some embodiments, the human subject is a female. In some embodiments, the human subject is a male.

Symptoms of PSC include, but are not limited to, jaundice, non-specific upper abdominal pain, fatigue, pruritus, right upper quadrant, pain yellowing of the skin, mucous membranes, and whites of the eyes (obstructive jaundice). Additional symptoms include a general feeling of ill health (malaise); abdominal pain, especially the upper right portion of the abdomen; nausea; dark urine; light-colored stools; unintended weight loss, and/or abnormal enlargement of the liver (hepatomegaly) and/or spleen (splenomegaly).

In any of the embodiments described herein, the agonist of the MST1/MST1R pathway is an MST1 agonist or an MST1R agonist. In some embodiments, the MST1 agonist and/or the MST1R agonist comprises a protein, a nucleic acid molecule, or a small molecule. In some embodiments, the protein is an antibody to MST1 or MST1R.

In some embodiments, the MST1 agonist is a protein, such as recombinant MST1. In some embodiments, the recombinant MST1 is a fusion protein comprising MST1 or a fragment thereof fused to a heterologous protein, for example an antibody or a fragment thereof, such as Fc fragment (e.g., Mst1-Fc). In some embodiments, the MST1 agonist is chelerythrine, recombinant Tumor Necrosis Factor Receptor Associated Factor 2 (TRAF2), or curcumin. In some embodiments, the MST1 agonist is a protease chosen from Hepatocyte Growth Factor Activator (HGFA), matriptase, hepsin, TMPRSS11D (Human Airway Prypsin-like protease; HAT), clotting factor XIIa, clotting factor Xia, and kallikrein.

In some embodiments, the MST1R agonist is recombinant Macrophage Scavenger Receptor 1 (MSR1), recombinant Hepatocyte Growth Factor-Like protein (HGFL), or recombinant Androgen Receptor (AR). In some embodiments, the MST1R agonist is an antibody chosen from mAb Zt/g4, mAb Zt/c1, mAb Zt/f2, mAb Zt/64, mAb 3F12, mAb B9, and mAb 1G4.

In any of the embodiments described herein, the patient having IBD and/or PSC can be treated with additional therapeutic agents. Examples of therapeutic agents that treat or inhibit IBD include, but are not limited to, aminosalicylates (such as COLAZAL® (balsalazide), ASACOL®, APRISO®, LIALDA®, and PENTASA® (mesalamine), DIPENTUM® (olsalazine), and AZULFIDINE® (sulfasalazine)); corticosteroids; immune modifying agents (such as IMURAN® (azathioprine), RHEUMATREX® (methotrexate), and PURINETHOL® (6-mercaptopurine (6-MP)); and antibodies (such as REMICADE® (infliximab), RENFLEXIS® (infliximab-abda), INFLECTRA® (infliximab-dyyb), HUMIRA® (dalimumab), AMJEVITA® (adalimumab-atto), CIMZIA® (certolizumab), TYSABRI® (natalizumab), ENTYVIO® (vedolizumab), STELARA® (ustekinumab), SIMPONI® and SIMPONI® ARIA (golimumab)), and recombinant MST1, or any combination thereof. Examples of therapeutic agents that treat or inhibit IBD include, but are not limited to, aminosalicylates (such as balsalazide, mesalamine, olsalazine, and sulfasalazine); corticosteroids; immune modifying agents (such as azathioprine, methotrexate, and 6-mercaptopurine (6-MP)); and antibodies (such as infliximab, infliximab-abda, infliximab-dyyb, dalimumab, adalimumab-atto, certolizumab, natalizumab, vedolizumab, ustekinumab, golimumab), and recombinant MST1, or any combination thereof. Examples of therapeutic agents that treat or inhibit PSC include, but are not limited to, medications to reduce itching and jaundice, antibiotics to treat infections, immunosuppressants, cholecystogues, and vitamin supplements, or any combination thereof.

Administration of the therapeutic agents that treat or inhibit IBD or PSC can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a patient can receive therapy for a prolonged period of time such as, for example, 6 months, 1 year, or more.

Administration of the therapeutic agents that treat or inhibit IBD or PSC can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in IBD, PSC, or both, a decrease/reduction in the severity of IBD, PSC, or both (such as, for example, a reduction or inhibition of development of IBD, PSC, or both), a decrease/reduction in symptoms and IBD-related effects, PSC-related effects, or both, delaying the onset of symptoms and IBD-related effects, PSC-related effects, or both, reducing the severity of symptoms of IBD-related effects, PSC-related effects, or both, reducing the severity of an acute episode, reducing the number of symptoms and IBD-related effects, PSC-related effects, or both, reducing the latency of symptoms and IBD-related effects, PSC-related effects, or both, an amelioration of symptoms and IBD-related effects, PSC-related effects, or both, reducing secondary symptoms, reducing secondary infections, preventing relapse to IBD, PSC, or both, decreasing the number or frequency of relapse episodes, increasing latency between symptomatic episodes, increasing time to sustained progression, speeding recovery, and/or increasing efficacy of or decreasing resistance to alternative therapeutics, following administration of the agent or composition comprising the agent. A prophylactic effect may comprise a complete or partial avoidance/inhibition or a delay of IBD, PSC, or both development/progression (such as, for example, a complete or partial avoidance/inhibition or a delay) following administration of a therapeutic protocol. Treatment of IBD, PSC, or both encompasses the treatment of patients already diagnosed as having any form of IBD, PSC, or both at any clinical stage or manifestation, the delay of the onset or evolution or aggravation or deterioration of the symptoms or signs of IBD, PSC, or both, and/or preventing and/or reducing the severity of IBD, PSC, or both.

In any of the embodiments described herein, the methods can further comprise detecting the presence or absence of an MST1 and/or MST1R variant nucleic acid molecule or variant polypeptide associated with an increased risk of developing IBD and/or PSC in a biological sample from the patient. It is understood that gene sequences within a population and mRNA molecules encoded by such genes can vary due to polymorphisms such as single-nucleotide polymorphisms. The sequences provided herein for the MST1 and/or MST1R variant nucleic acid molecules disclosed herein are only exemplary sequences. Other sequences for the MST1 and/or MST1R variant nucleic acid molecules are also possible.

The MST1 and/or MST1R variant nucleic acid molecule or variant polypeptide associated with an increased risk of developing IBD and/or PSC can be any MST1 and/or MST1R predicted loss-of-function variant or MST1 and/or MST1R missense variant, such as any of those described herein. For example, in any of the embodiments described herein, the MST1 variant nucleic acid molecule associated with an increased risk of developing IBD and/or PSC is Arg703Cys or Arg651STOP. In some embodiments, the MST1 variant nucleic acid molecule associated with an increased risk of developing IBD and/or PSC is Arg703Cys. In some embodiments, the MST1 variant nucleic acid molecule associated with an increased risk of developing IBD and/or PSC is Arg651STOP. In addition, in any of the embodiments described herein, the MST1R variant nucleic acid molecule associated with an increased risk of developing IBD and/or PSC can be 3:49903264:CG:C, 3:49903084:C:T, 3:49890026:G:T, 3:49902417:A:G, 3:49902560:A:T, or 3:49903387:G:T, or an mRNA molecule produced therefrom, or a cDNA molecule produced from the mRNA molecule. In any of the embodiments described herein, the MST1R variant nucleic acid molecule associated with an increased risk of developing IBD and/or PSC is 3:49903264:CG:C, or an mRNA molecule produced therefrom, or a cDNA molecule produced from the mRNA molecule. In any of the embodiments described herein, the MST1R variant nucleic acid molecule associated with an increased risk of developing IBD and/or PSC is 3:49903084:C:T, or an mRNA molecule produced therefrom, or a cDNA molecule produced from the mRNA molecule. In any of the embodiments described herein, the MST1R variant nucleic acid molecule associated with an increased risk of developing IBD and/or PSC is 3:49890026:G:T, or an mRNA molecule produced therefrom, or a cDNA molecule produced from the mRNA molecule. In any of the embodiments described herein, the MST1R variant nucleic acid molecule associated with an increased risk of developing IBD and/or PSC is 3:49902417:A:G, or an mRNA molecule produced therefrom, or a cDNA molecule produced from the mRNA molecule. In any of the embodiments described herein, the MST1R variant nucleic acid molecule associated with an increased risk of developing IBD and/or PSC is 3:49902560:A:T, or an mRNA molecule produced therefrom, or a cDNA molecule produced from the mRNA molecule. In any of the embodiments described herein, the MST1R variant nucleic acid molecule associated with an increased risk of developing IBD and/or PSC can be 3:49903387:G:T, or an mRNA molecule produced therefrom, or a cDNA molecule produced from the mRNA molecule.

In some embodiments, detecting the presence or absence of the MST1 and/or MST1R variant nucleic acid molecule or variant polypeptide associated with an increased risk of developing IBD and/or PSC comprises determining whether the patient has an MST1 and/or MST1R variant genomic nucleic acid molecule associated with an increased risk of developing IBD and/or PSC, an MST1 and/or MST1R variant mRNA molecule associated with an increased risk of developing IBD and/or PSC, an MST1 and/or MST1R variant cDNA molecule produced from the mRNA molecule, and/or an MST1 and/or MST1R variant polypeptide associated with an increased risk of developing IBD and/or PSC. In some embodiments, such determination is carried out by obtaining or having obtained a biological sample from the patient, and performing or having performed an assay on the biological sample to determine whether the patient has an MST1 and/or MST1R variant nucleic acid molecule or variant polypeptide associated with an increased risk of developing IBD and/or PSC. In some embodiments, the MST1 variant nucleic acid molecule is Arg703Cys or Arg651STOP. In some embodiments, the MST1 variant nucleic acid molecule is

Arg703Cys. In some embodiments, the MST1 variant nucleic acid molecule is Arg651STOP. In some embodiments, the MST1R variant nucleic acid molecule is any of the MST1R variant nucleic acid molecules described herein.

Determining whether a human has an MST1 and/or MST1R variant nucleic acid molecule or an MST1 and/or MST1R variant polypeptide associated with an increased risk of developing IBD and/or PSC in a biological sample can be carried out by any of the methods described herein. In some embodiments, the detecting step, determining step, or assay is carried out in vitro. In some embodiments, these methods can be carried out in situ. In some embodiments, these methods can be carried out in vivo. In any of these embodiments, the nucleic acid molecule can be present within a cell obtained from the human subject. In some embodiments, the assay is a genotyping assay for nucleic acid molecules. In some embodiments, the assay is an immunoassay for polypeptides.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. In some embodiments, the biological sample comprises a cell lysate. Such methods can further comprise obtaining a biological sample from the subject. The sample used in the methods disclosed herein will vary based on the assay format, nature of the detection method, and the tissues, cells, or extracts that are used as the sample. A biological sample can be processed differently depending on the assay being employed. For example, when detecting any MST1 and/or MST1R variant nucleic acid molecule, preliminary processing designed to isolate or enrich the sample for the genomic DNA can be employed. A variety of known techniques may be used for this purpose. When detecting the level of any MST1 and/or MST1R variant mRNA, different techniques can be used enrich the biological sample with mRNA. Various methods to detect the presence or level of a mRNA or the presence of a particular variant genomic DNA locus can be used.

In some embodiments, the methods can further comprise determining the patient's aggregate burden of having MST1 and/or MST1R variant genomic nucleic acid molecules associated with an increased risk of developing IBD and/or PSC, MST1 and/or MST1R variant mRNA molecules associated with an increased risk of developing IBD and/or PSC, MST1 and/or MST1R variant cDNA molecules produced from the mRNA molecules, and/or MST1 and/or MST1R variant polypeptides associated with an increased risk of developing IBD and/or PSC. The aggregate burden is the sum of all rare variants in the MSTR1 gene, which is run in an association test with IBD. The result of the association test suggests that rare loss-of-function and missense variants of MST1R are associated with increased odds of IBD. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises sequencing at least a portion of the nucleotide sequence of the MST1 and/or MST1R nucleic acid molecule in the biological sample. The sequenced portion comprises a position corresponding to a predicted loss-of-function variant position. When a variant nucleotide at the predicted loss-of-function variant position is detected, the MST1 and/or MST1R nucleic acid molecule in the biological sample is an MST1 and/or MST1R predicted loss-of-function variant nucleic acid molecule. The predicted loss-of-function variant position within any particular MST1 and/or MST1R nucleic acid molecule is the one or more positions of the variant nucleotide sequence that are different compared to the nucleotide sequence of the corresponding reference nucleic acid molecule. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises: contacting the biological sample with a primer hybridizing to a portion of the nucleotide sequence of the MST1 and/or MST1R nucleic acid molecule that is proximate to a predicted loss-of-function variant position, extending the primer at least through the predicted loss-of-function variant position, and determining whether the extension product of the primer comprises a variant nucleotide at the predicted loss-of-function variant position. In some embodiments, the detecting step, determining step, or assay comprises sequencing the entire nucleic acid molecule. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises sequencing at least a portion of the nucleotide sequence of the MST1 and/or MST1R nucleic acid molecule in the biological sample. The sequenced portion comprises a position corresponding to a missense variant position. When a variant nucleotide at the missense variant position is detected, the MST1 and/or MST1R nucleic acid molecule in the biological sample is an MST1 and/or MST1R missense variant nucleic acid molecule. The missense variant position within any particular MST1 and/or MST1R nucleic acid molecule is the one or more positions of the variant nucleotide sequence that are different compared to the nucleotide sequence of the corresponding reference nucleic acid molecule. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises contacting the biological sample with a primer hybridizing to a portion of the nucleotide sequence of the MST1 and/or MST1R nucleic acid molecule that is proximate to a missense variant position, extending the primer at least through the missense variant position, and determining whether the extension product of the primer comprises a variant nucleotide at the missense variant position. In some embodiments, the detecting step, determining step, or assay comprises sequencing the entire nucleic acid molecule. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the assay comprises contacting the biological sample with a primer, such as an alteration-specific primer, that specifically hybridizes to an MST1 or MST1R variant genomic sequence, variant mRNA sequence, or variant cDNA sequence and not the corresponding MST1 or MST1R reference sequence under stringent conditions. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, only an MST1 and/or MST1R genomic nucleic acid molecule is analyzed. In some embodiments, only an MST1 and/or MST1R mRNA is analyzed. In some embodiments, only an MST1 and/or MST1R cDNA obtained from MST1 and/or MST1R mRNA is analyzed. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

Illustrative examples of nucleic acid sequencing techniques include, but are not limited to, chain terminator (Sanger) sequencing and dye terminator sequencing. In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the detecting step, determining step, or assay comprises amplifying at least a portion of the MST1 and/or MST1R nucleic acid molecule, wherein the portion comprises a predicted loss-of-function variant position, labeling the amplified nucleic acid molecule with a detectable label, contacting the labeled nucleic acid molecule with a support comprising an alteration-specific probe, wherein the alteration-specific probe comprises a nucleotide sequence which hybridizes under stringent conditions to the predicted loss-of-function variant position, and detecting the detectable label. In some embodiments, the nucleic acid molecule in the sample is mRNA and the mRNA is reverse-transcribed into a cDNA prior to the amplifying step. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises contacting the nucleic acid molecule in the biological sample with an alteration-specific probe comprising a detectable label, wherein the alteration-specific probe comprises a nucleotide sequence which hybridizes under stringent conditions to a predicted loss-of-function variant position, and detecting the detectable label. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises amplifying at least a portion of the MST1 and/or MST1R nucleic acid molecule, wherein the portion comprises a missense variant position, labeling the amplified nucleic acid molecule with a detectable label, contacting the labeled nucleic acid molecule with a support comprising an alteration-specific probe, wherein the alteration-specific probe comprises a nucleotide sequence which hybridizes under stringent conditions to the missense variant position, and detecting the detectable label. In some embodiments, the nucleic acid molecule in the sample is mRNA and the mRNA is reverse-transcribed into a cDNA prior to the amplifying step. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the detecting step, determining step, or assay comprises contacting the nucleic acid molecule in the biological sample with an alteration-specific probe comprising a detectable label, wherein the alteration-specific probe comprises a nucleotide sequence which hybridizes under stringent conditions to a missense variant position, and detecting the detectable label. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The alteration-specific probes or alteration-specific primers described herein comprise a nucleic acid sequence which is complementary to and/or hybridizes, or specifically hybridizes, to an MST1 or MST1R predicted loss-of-function variant nucleic acid molecule, or an MST1 or MST1R missense variant nucleic acid molecule, or the complement thereof. In some embodiments, the alteration-specific probes or alteration-specific primers comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 nucleotides. In some embodiments, the alteration-specific probes or alteration-specific primers comprise or consist of at least 15 nucleotides. In some embodiments, the alteration-specific probes or alteration-specific primers comprise or consist of at least 15 nucleotides to at least about 35 nucleotides. In some embodiments, alteration-specific probes or alteration-specific primers hybridize to MST1 or MST1R predicted loss-of-function variant genomic nucleic acid molecules, MST1R predicted loss-of-function variant mRNA molecules, and/or MST1R predicted loss-of-function variant cDNA molecules under stringent conditions. In some embodiments, alteration-specific probes or alteration-specific primers hybridize to MST1 or MST1R missense variant genomic nucleic acid molecules, MST1R missense variant mRNA molecules, and/or MST1R missense variant cDNA molecules under stringent conditions. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the assay comprises contacting the biological sample with a probe, such as an alteration-specific probe, that specifically hybridizes to an MST1 or MST1R variant genomic sequence, variant mRNA sequence, or variant cDNA sequence and not the corresponding MST1 or MST1R reference sequence under stringent conditions, and determining whether hybridization has occurred. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

In some embodiments, the methods utilize probes and primers of sufficient nucleotide length to bind to the target nucleotide sequence and specifically detect and/or identify a polynucleotide comprising an MST1 or MST1R variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, labeled primers or probes directed against purified DNA, amplified DNA, and fixed cell preparations (fluorescence in situ hybridization (FISH)) can be used for detection. In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. Stringent conditions are sequence-dependent and will be different in different circumstances.

Appropriate stringency conditions which promote DNA hybridization, for example, 6×sodium chloride/sodium citrate (SSC) at about 45° C., followed by a wash of 2X SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, detecting the presence of a human MST1 and/or MST1R variant polypeptide comprises performing an assay on a sample obtained from a human subject to determine whether an MST1 and/or MST1R polypeptide in the subject contains one or more variations that causes the polypeptide to have a loss-of-function (partial or complete) or predicted loss-of-function (partial or complete), or be produced from a missense variant nucleic acid molecule. In some embodiments, the assay comprises sequencing at least a portion of the MST1 and/or MST1R polypeptide that comprises a variant position. In some embodiments, the detecting step comprises sequencing the entire polypeptide. Identification of a variant amino acid at the variant position of the MST1 and/or MST1R polypeptide indicates that the MST1 and/or MST1R polypeptide is an MST1 and/or MST1R predicted loss-of-function polypeptide, or is produced from a missense variant nucleic acid molecule. In some embodiments, the assay comprises an immunoassay for detecting the presence of a variant polypeptide. Detection of a variant amino acid at the variant position of the MST1 or MST1R polypeptide indicates that the MST1 or MST1R polypeptide is a variant MST1 or MST1R polypeptide. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The probes and/or primers (including alteration-specific probes and alteration-specific primers) described herein comprise or consist of from about 15 to about 100, from about 15 to about 35 nucleotides. In some embodiments, the alteration-specific probes and alteration-specific primers comprise DNA. In some embodiments, the alteration-specific probes and alteration-specific primers comprise RNA. In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers (including alteration-specific probes and alteration-specific primers) specifically hybridize to any of the nucleic acid molecules disclosed herein under stringent conditions. In the context of the disclosure “specifically hybridizes” means that the probe or primer (including alteration-specific probes and alteration-specific primers) does not hybridize to a nucleic acid sequence encoding an MST1R reference genomic nucleic acid molecule, an MST1R reference mRNA molecule, and/or an MST1R reference cDNA molecule. In some embodiments, the probes (such as, for example, an alteration-specific probe) comprise a label. In some embodiments, the label is a fluorescent label, a radiolabel, or biotin.

The present disclosure also provides methods of identifying a human subject having an increased risk of developing IBD. The methods comprise determining or having determined whether the subject has any one or more of the MST1R predicted loss-of-function variant nucleic acid molecule or polypeptide produced therefrom described herein. The methods can also comprise determining or having determined whether the subject has any one or more of the MST1R missense variant nucleic acid molecule or polypeptide produced therefrom described herein. When the subject has an MST1R predicted loss-of-function variant nucleic acid molecule, or an MST1R missense variant nucleic acid molecule, or polypeptide produced therefrom, the subject has an increased risk of developing IBD. In some embodiments, when a human subject is identified as having an increased risk of developing IBD, the human subject is further treated with a therapeutic agent that treats or inhibits IBD and/or an MST1/MST1R pathway agonist, as described herein. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The present disclosure also provides methods of identifying a human subject having an increased risk of developing PSC. The methods comprise determining or having determined whether the subject has any one or more of the MST1R predicted loss-of-function variant nucleic acid molecule or polypeptide produced therefrom described herein. The methods can also comprise determining or having determined whether the subject has any one or more of the MST1R missense variant nucleic acid molecule or polypeptide produced therefrom described herein. When the subject has an MST1R predicted loss-of-function variant nucleic acid molecule, or an MST1R missense variant nucleic acid molecule, or polypeptide produced therefrom, the subject has an increased risk of developing PSC. In some embodiments, when a human subject is identified as having an increased risk of developing PSC, the human subject is further treated with a therapeutic agent that treats or inhibits PSC and/or an MST1/MST1R pathway agonist, as described herein. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The present disclosure also provides methods of diagnosing IBD in a human subject. The methods comprise determining or having determined whether the subject has any one or more of the MST1R predicted loss-of-function variant nucleic acid molecule or polypeptide produced therefrom described herein. The methods can also comprise determining or having determined whether the subject has any one or more of the MST1R missense variant nucleic acid molecule or polypeptide produced therefrom described herein. When the subject has an MST1R predicted loss-of-function variant nucleic acid molecule, or an MST1R missense variant nucleic acid molecule, or polypeptide produced therefrom, and has one or more symptoms of IBD, the subject is diagnosed as having IBD. In some embodiments, when a human subject is identified as having IBD, the human subject is further treated with a therapeutic agent that treats or inhibits IBD and/or an MST1/MST1R pathway agonist, as described herein. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The present disclosure also provides methods of diagnosing PSC in a human subject. The methods comprise determining or having determined whether the subject has any one or more of the MST1R predicted loss-of-function variant nucleic acid molecule or polypeptide produced therefrom described herein. The methods can also comprise determining or having determined whether the subject has any one or more of the MST1R missense variant nucleic acid molecule or polypeptide produced therefrom described herein. When the subject has an MST1R predicted loss-of-function variant nucleic acid molecule, or an MST1R missense variant nucleic acid molecule, or polypeptide produced therefrom, and has one or more symptoms of IBD, the subject is diagnosed as having PSC. In some embodiments, when a human subject is identified as having PSC, the human subject is further treated with a therapeutic agent that treats or inhibits PSC and/or an MST1/MST1R pathway agonist, as described herein. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

The present disclosure also provides molecular complexes comprising any of the MST1R variant nucleic acid molecules (genomic nucleic acid molecules, mRNA molecules, or cDNA molecules), or complement thereof, described herein and any of the alteration-specific primers or alteration-specific probes described herein. In some embodiments, the MST1R variant nucleic acid molecules (genomic nucleic acid molecules, mRNA molecules, or cDNA molecules), or complement thereof, in the molecular complexes are single-stranded. In some embodiments, the MST1R variant nucleic acid molecule is any of the variant genomic nucleic acid molecules described herein. In some embodiments, the MST1R variant nucleic acid molecule is any of the variant mRNA molecules described herein. In some embodiments, the MST1R variant nucleic acid molecule is any of the variant cDNA molecules described herein. In some embodiments, the molecular complex comprises any of the MST1R variant nucleic acid molecules (genomic nucleic acid molecules, mRNA molecules, or cDNA molecules), or complement thereof, described herein and any of the alteration-specific primers described herein. In some embodiments, the molecular complex comprises any of the MST1R variant nucleic acid molecules (genomic nucleic acid molecules, mRNA molecules, or cDNA molecules), or complement thereof, described herein and any of the alteration-specific probes described herein. In some embodiments, the molecular complex comprises a non-human polymerase. In some embodiments, the MST1 variant nucleic acid molecule is Arg703Cys or Arg651STOP. In some embodiments, the MST1 variant nucleic acid molecule is Arg703Cys. In some embodiments, the MST1 variant nucleic acid molecule is Arg651STOP. In some embodiments, the MST1R variant nucleic acid molecule is any of the MST1R variant nucleic acid molecules described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Aggregate Burden

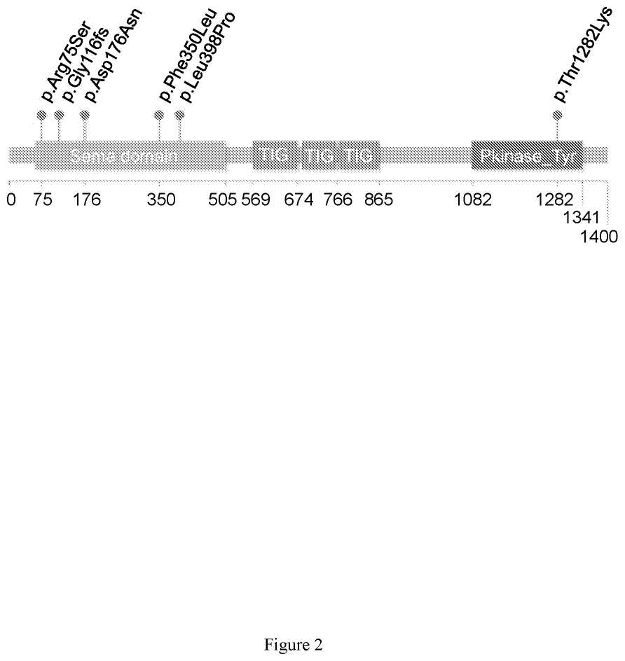

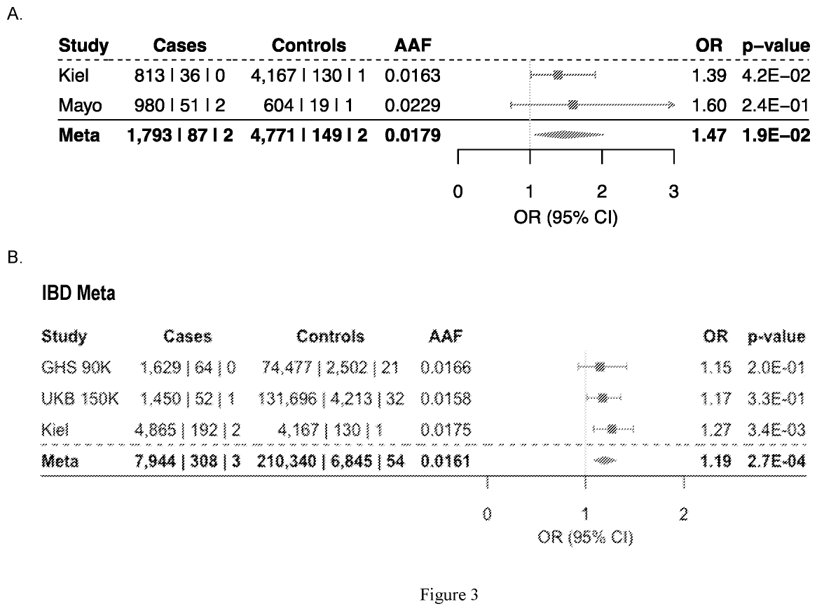

Table 1 shows that the aggregate of all rare predicted loss-of-function (pLOF) and predicted missense variants significantly associates with increased odds of IBD among exome-wide analysis of 4,319 IBD cases and 4,388 cases within the University of Kiel IBD cohort (odds ratio 2.99; p-value 1.62E-06). Table 1 also shows that the aggregate of all rare predicted loss-of-function variants associates with increased odds of IBD among exome-wide analysis of 4,319 IBD cases and 4,388 cases within the University of Kiel IBD cohort (odds ratio 3.90; p-value 2.15E-04) (see also, FIG. 2).

TABLE 1

| | # | Case | # | Control | | OR | |
|---|---|---|---|---|---|---|---|
| Mask | Cases | RR:RA:AA | Controls | RR:RA:AA | AAF | (95% CI) | P-value |
| pLoF and | 4319 | 4234:85:0 | 4388 | 4359:29:0 | 0.0065 | 2.99 | 1.62E−06 |
| predicted | | | | | | (1.91, 4.68) | |
| deleterious | | | | | | | |
| missense | | | | | | | |
| variants | | | | | | | |
| (M3.1) | | | | | | | |
| pLoF | 4319 | 4283:36:0 | 4388 | 4380:8:0 | 0.0025 | 3.90 | 2.15E−04 |
| variants | | | | | | (1.89, 8.03) | |
| (M1.1) | | | | | | | |

Table 2 shows that the association of the aggregate of all predicted loss-of-function variants replicates in UK Biobank IBD cohort comprised of 396 cases and 137,000 controls at an odds ratio of 2.22 (p-value 0.018) and 3.21 (p-value 0.011), respectively. Table 2 also shows that the association of the aggregate of all rare predicted loss-of-function and predicted missense variants and the aggregate of all rare predicted loss-of-function variants replicates in UK Biobank IBD cohort comprised of 494 cases and 30,000 controls at an odds ratio of 2.21 (p-value 0.048).

TABLE 2

| | | Case | Control | | OR | |
|---|---|---|---|---|---|---|
| Cohort | Mask | RR:RA:AA | RR:RA:AA | AAF | (95% CI) | P-value |
| UKB | pLoF and | 387:9:0 | 136174:1414:0 | 0.0190 | 2.22 | 1.8E−2 |
| 150K | predicted | | | | (1.15, 4.32) | |
| | deleterious | | | | | |
| | missense | | | | | |
| | variants | | | | | |
| | (M3.1) | | | | | |
| | pLoF | 391:5:0 | 137044:544:0 | 0.0035 | 3.21 | 1.1E−2 |
| | variants | | | | (1.30, 7.90) | |
| | (M1.1) | | | | | |
| GHS | pLoF | 488:6:0 | 30978:189:0 | 0.0031 | 2.21 | 4.8E−2 |
| 60K | variants | | | | (1.01, 4.87) | |
| | (M1.1) | | | | | |

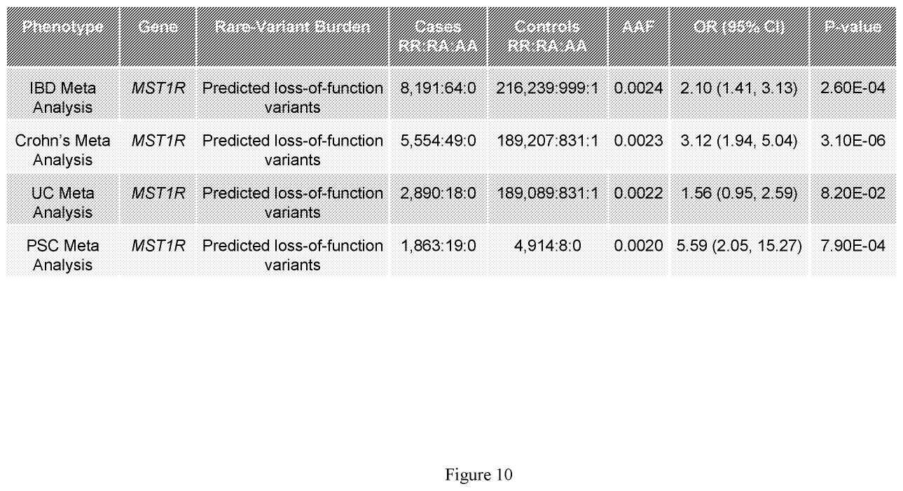

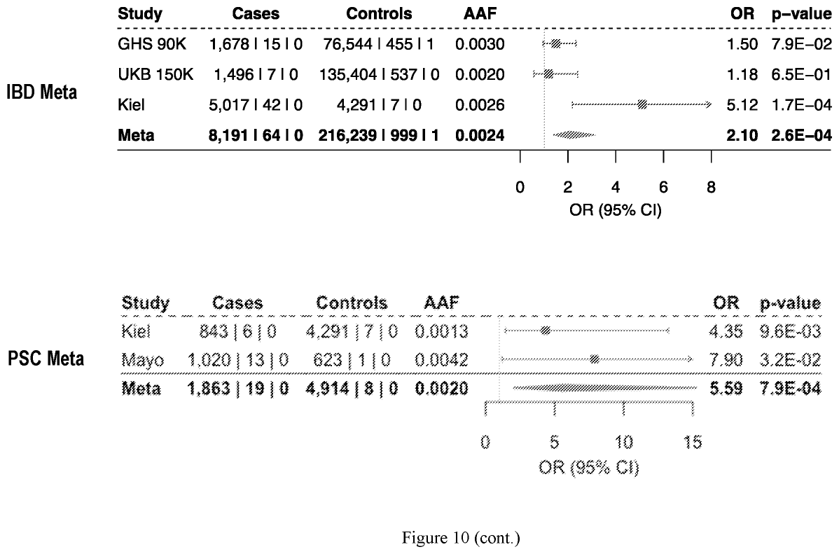

Meta-analysis confirmed this large-effect, rare-variant associations between rare, loss-of-function variants in MST1R to Crohn's (OR=3.12) and PSC (OR=5.59) (see, FIG. 10).

The association of the individual MST1R variants with Crohn's disease is shown in Table 3.

TABLE 3

| | rsID | Case | Control | CD OR | | |
|---|---|---|---|---|---|---|
| Variant | HGVS p. | RR:RA:AA | RR:RA:AA | (95% CI) | p-value | AAF |
| 3:49903264:CG:C | rs758062275 | 4,307:13:0 | 4,388:0:0 | 27.47 | 6.30E−04 | 0.0007 |
| | p.Gly116fs | | | (4.11, 183.49) | | |
| 3:49903084:C:T | rs151106960 | 4,310:10:0 | 4,386:2:0 | 4.27 | 5.56E−02 | 0.0007 |
| | p.Asp176Asn | | | (0.97, 18.85) | | |
| 3:49890026:G:T | rs369707898 | 4,316:4:0 | 4,387:1:0 | 3.05 | 2.38E−01 | 0.0003 |
| | p.Thr1282Lys | | | (0.48, 19.18) | | |
| 3:49902417:A:G | rs199980524 | 4,316:4:0 | 4,386:2:0 | 1.83 | 3.41E−01 | 0.0003 |
| | p.Leu398Pro | | | (0.53, 6.30) | | |
| 3:49902560:A:T | rs199531010 | 4,317:3:0 | 4,385:3:0 | 1.02 | 5.68E−01 | 0.0003 |
| | p.Phe350Leu | | | (0.96, 1.07) | | |
| 3:49903387:G:T | rs35887539 | 4,317:3:0 | 4,386:2:0 | 1.42 | 8.71E−01 | 0.0003 |
| | p.Arg75Ser | | | (0.02, 98.94) | | |

### Example 2: Mouse Model of IBD (Prophetic Example)

The effect of the loss of MST1R signaling on the increased risk in IBD mouse models will also be determined. MST1R KO mice will be generated, and challenged in mouse models of IBD. It is anticipated that mice lacking MST1R will be more susceptible to mouse models of IBD. In addition, MST1R transgenic mice that have constitutive MST1R signaling will be generated. It is anticipated that these mice will also be protected in mouse models of IBD.

In vitro experiments will also be performed to determine whether MST1 protein expressing rs3197999 will bind and signal through MST1R. Recombinant protein expressing the MST1 missense rs3197999 will be generated, and macrophage cell lines expressing normal MST1R will be treated with this protein and wildtype protein. Through measurements of signaling downstream of MST1R phospho-AKT/PI3K signaling, it will be determined whether rs3197999 MST1 protein is able to activate MST1R signaling as compared to wildtype MST1 protein. In addition, macrophage cell lines that express MST1R loss-of-function/missense variants will be generated to determine if these mutant proteins can signal by measuring downstream phospho-AKT/PI3K signaling molecules in the presence of exogenous wildtype MST1 ligand.

### Example 3: Novel MST1pLoF Mutation is Associated with IBD and PSC

A common MST1 missense variant (r53197999) has been associated with increased odds of IBD and PSC; MST1 rs3197999/p.Arg703Cys (29% allele frequency) has been associated with reduced serum MST1 protein levels (Table 4).

TABLE 4

| | Cases | Controls | Het OR | Hom OR | |
|---|---|---|---|---|---|
| Phenotype | RR:RA:AA | RR:RA:AA | (95% CI) | (95% CI) | p-value |
| IBD Meta | 6,009:4922:1137 | 110,756:86,514:16,720 | 1.07 | 1.37 | 9.40E−05 |
| Analysis | | | (1.02, 1.12) | (1.27, 1.48) | |
| Crohn's Meta | 2,719:2,303:581 | 98,121:76,930:14,988 | 1.08 | 1.40 | 2.40E−08 |
| Analysis | | | (1.02, 1.14) | (1.27, 1.53) | |
| UC Meta | 1,430:1,181:297 | 98,061:76,888:14,972 | 1.05 | 1.36 | 1.20E−04 |
| Analysi | | | (0.97, 1.14) | (1.20, 1.54) | |
| PSC Meta | 817:822:243 | 2,542:2,006:374 | 1.27 | 2.02 | 9.90E−07 |
| Analysis | | | (1.14, 1.43) | (1.68, 2.43) | |

A novel MST1 pLoF mutation (3:49684379:G:A generating a Stop gain; c.1951C>T; p.Arg651*) associating with a larger effect on PSC and IBD disease risk has been identified (Table 5 and FIG. 3), confirming that LoF of MST1 increased disease risk. This effect was greater than that of the MST1 missense mutation.

TABLE 5

| | Case | Control | OR | | |
|---|---|---|---|---|---|
| Cohort | RR:RA:AA | RR:RA:AA | (95% CI) | p-value | AAF |
| PSC Meta | 1793:87:2 | 4771:149:2 | 1.47 | 1.90E−2 | 0.018 |
| | | | (1.06, 2.04) | | |
| IBD Meta | 11617:447:4 | 207211:6726:53 | 1.19 | 2.7E−4 | 0.016 |
| | | | (1.09, 1.31) | | |

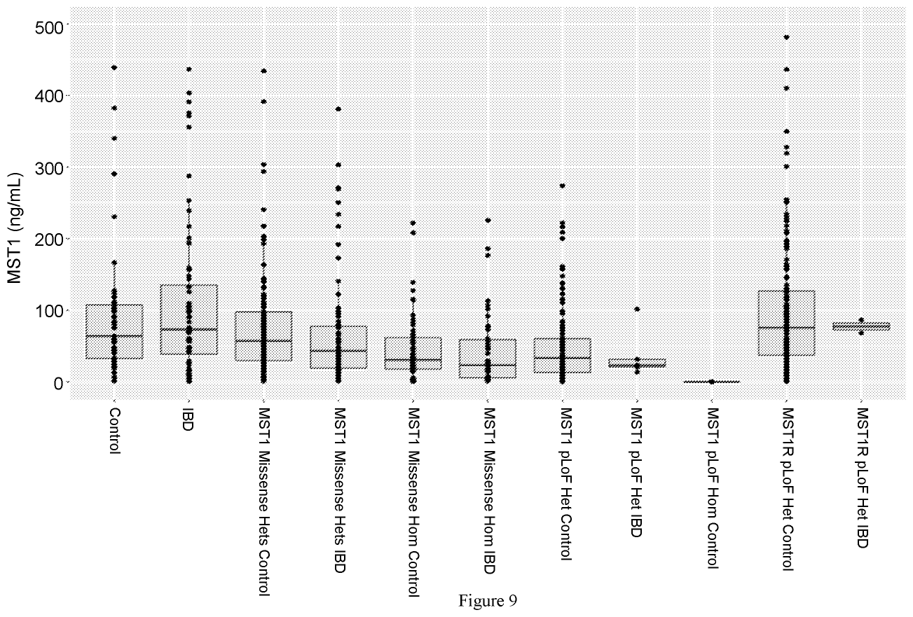

Serum levels of MST1 were measured in MST1 variant carriers and controls. Serum was obtained from heterozygous and homozygous carriers of MST1 p.R703C, heterozygous and homozygous MST1 p.651*, and from reference carriers (who carried neither MST1 p.R703C or MST1 p.651* variants). Circulating serum MST1 levels were measured using a Luminex assay. The serum levels of carriers of variant MST1 proteins were analyzed. The results show that both MST1 p.R703C missense carriers and novel p.651* pLoF variant carriers have significantly decreased levels of MST1 in serum relative to control reference carriers (FIG. 9). The assay that was used is disclosed at the world wide web at “rndsystems.com/products/human-magnetic-luminex-assay_lxsahm”.

### Example 4: Gastrointestinal Disorders Result in Changes of MST1 and MST1R Expression in Mice and in Human patients

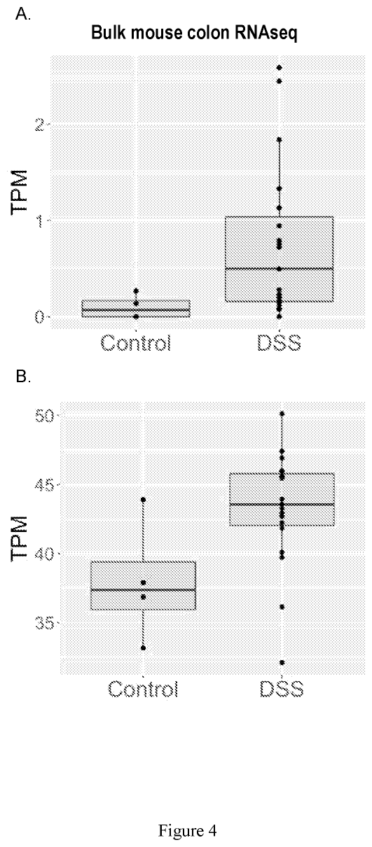

The levels of MST1 and MST1R mRNA were examined in mice that have been treated with DSS in order to induce colitis. Bulk RNAseq assays revealed that both MST1 and MST1R expression increase in mouse models of colitis (FIG. 4).

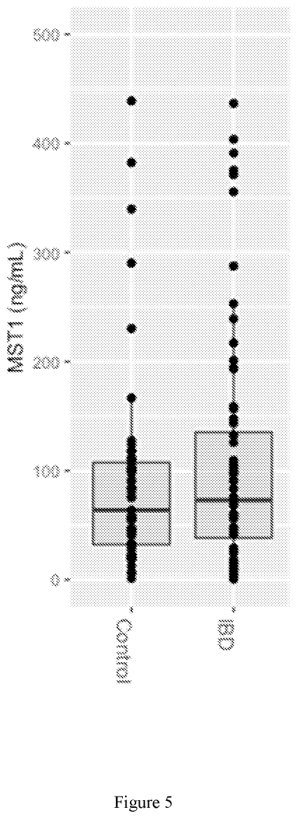

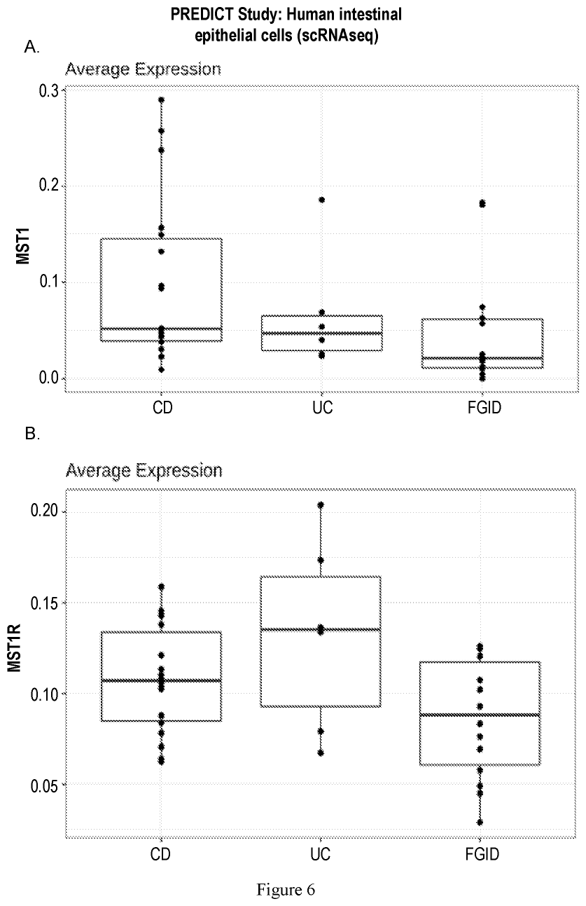

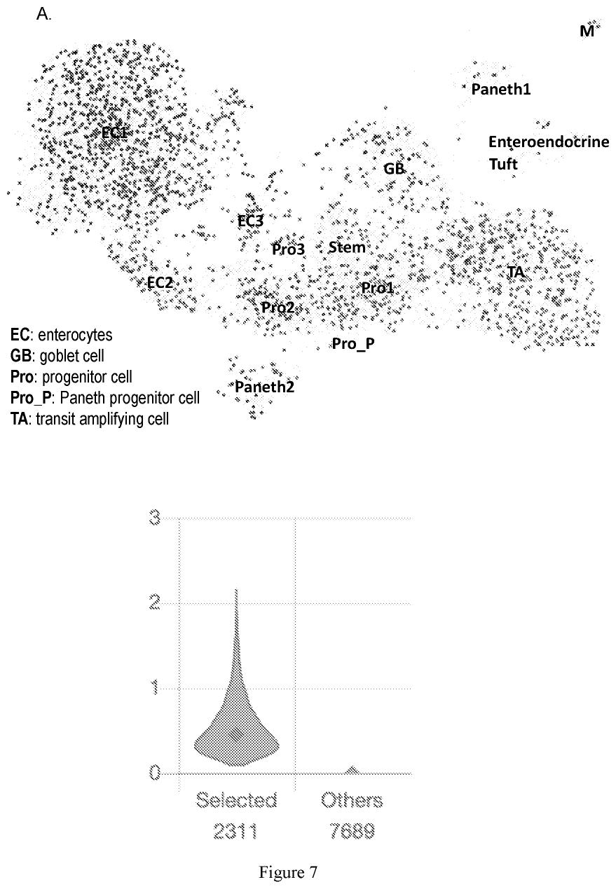

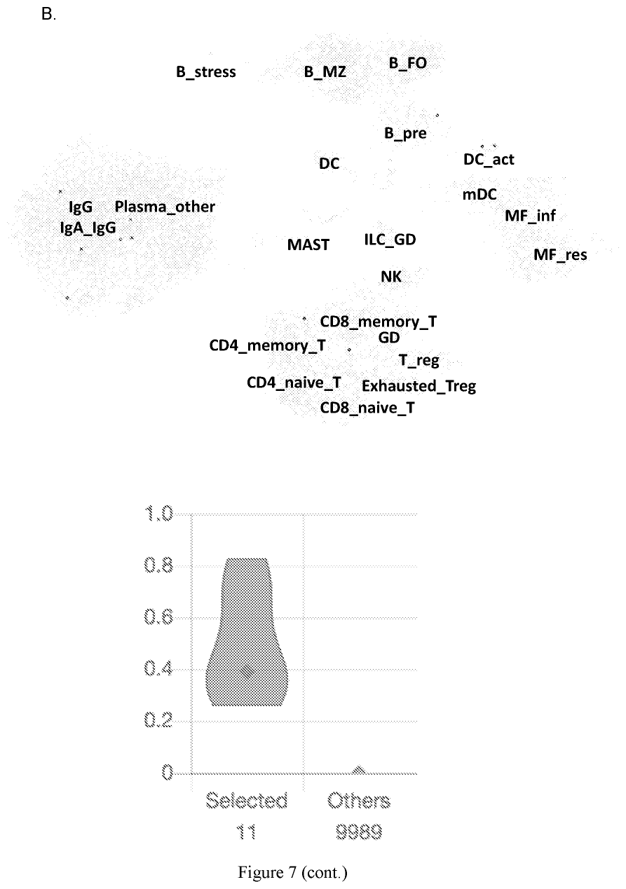

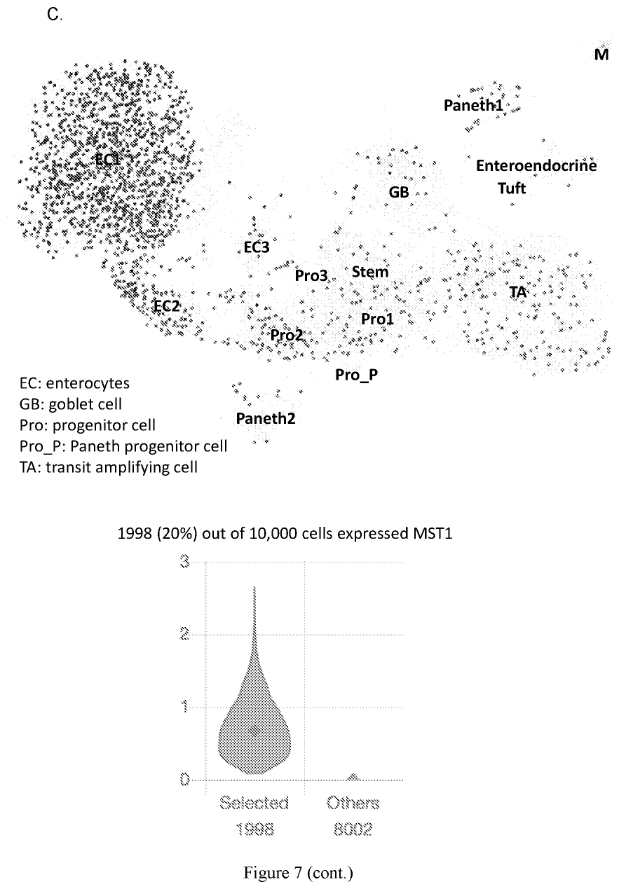

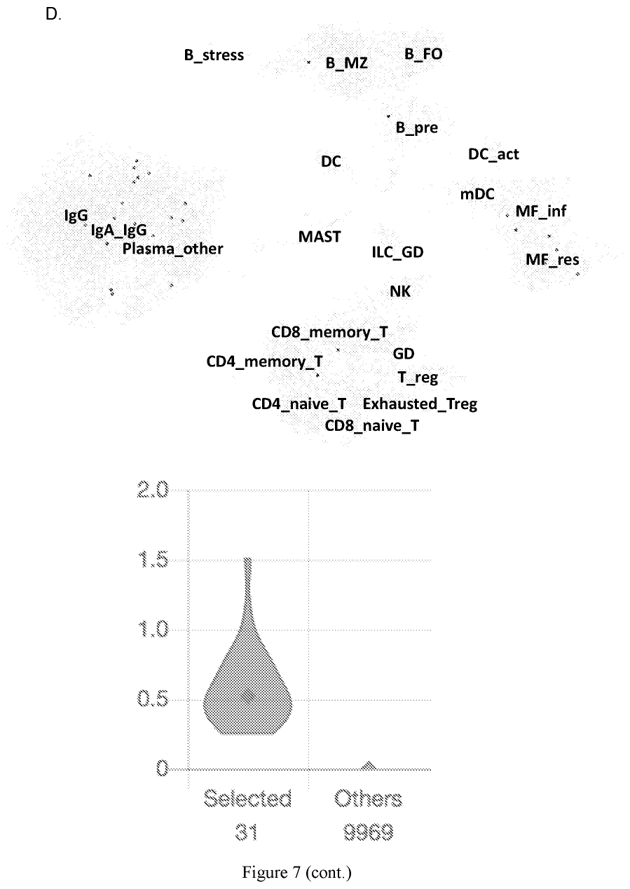

MST1 serum levels were assessed in human IBD patients as compared with healthy controls (FIG. 5). Serum was obtained from IBD patients and healthy controls (who did not carry MST1 p.R703C, MST1 p.651*, or MST1R pLOF variants), and MST1 was measured using a Luminex cytokine assay (as above). Analysis of these samples show an increase in MST1 serum concentration between IBD patients compared to healthy controls. MST1 and MST1R also increased in Crohn's disease, ulcerative colitis, and functional gastrointestinal disorder (FGID) patients (FIG. 6). Single cell RNA sequencing analysis was performed on intestinal biopsies of IBD patients with either Crohn's disease (CD), ulcerative colitis (UC), or patients with FGID as controls. Both MST1 and MST1R showed higher expression in small intestine epithelial cells of a human CD patient than in intestine lamina propria cells of a human FGID patient (FIG. 7).

In contrast to the increased wild type MST1/MST1R levels observed in IBD patients, the IBD patients carrying variant MST1 or MST1R showed decreased serum MST1 levels (FIG. 9). There was no difference between IBD and control levels among the variant carriers.

### Example 5: Expression of Variant MST1R Constructs

To ascertain whether an ATG codon encoding methionine at the position 464 of MST1R protein could be used as a translation start codon, expression of an MST1R cDNA construct carrying a G116fs mutation was examined. Western blot analysis of a HEK cell line transduced with a cDNA encoding either a full-length, wildtype MST1R or MST1R G116fs showed introduction of the frameshift in the cDNA of MST1R resulted in the translation of a short, truncated protein showing that downstream, in-frame ATG was not activated (FIG. 11).

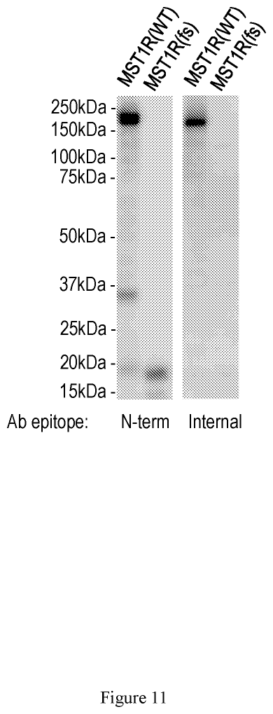

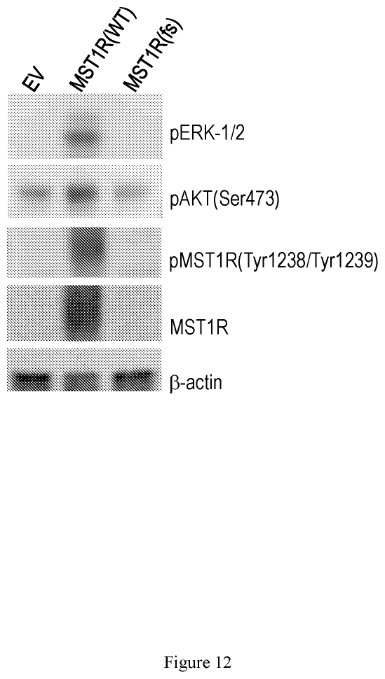

To analyze the properties of the truncated MST1R pLoF protein, HEK cells were transfected with constructs encoding either WT MST1R cDNA or a cDNA encoding an MST1R carrying the frameshift G116fs mutation. Western blot analysis of lysates generated from these cell lines show that while WT MST1R was robustly expressed and activated its downstream signaling targets, including phosphorylated ERK1/2 (pERK1/2), phosphorylated AKT (pAKT), and phosphorylated MST1R (pMST1R), no expression of mutant MST1R was detected and no activation of downstream targets was observed (FIG. 12), demonstrating that the MST1Rfs mutant is a true loss of function mutant.

### Example 6: Expression of Variant MST1 Constructs

The results of Example 1 show that the aggregate of rare pLoFs/missense variants in MST1R associates with increased risk of IBD, suggesting loss of MST1R increases the risk of IBD. It may be that increased MST1 protein levels and/or increased MST1/MST1R signaling is protective and/or promotes tissue repair in IBD and PSC.

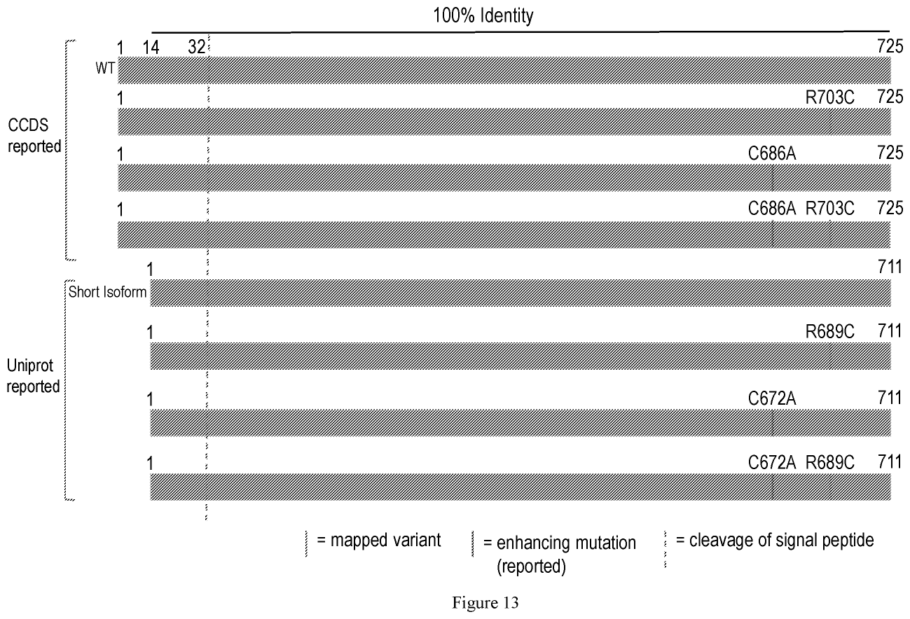

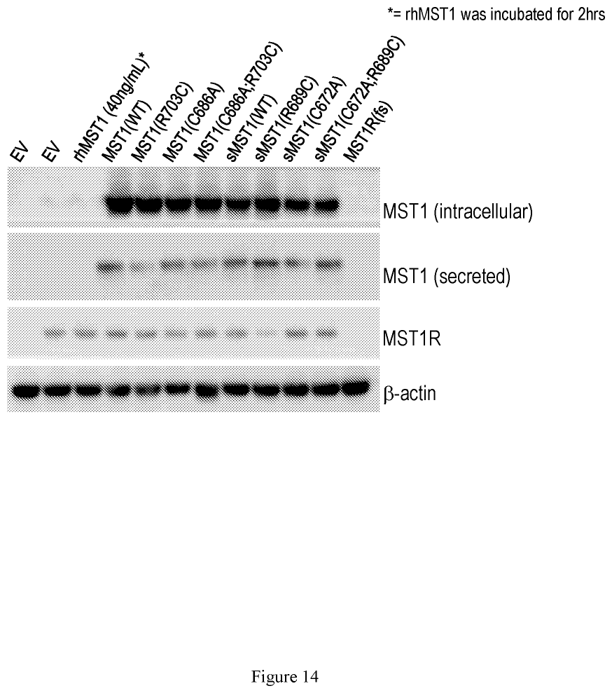

To gauge the possibility of MST1 expression, several constructs encoding WT and variant MST1 cDNAs (FIG. 13) were transfected into HEK cells. Western blot analyses showed that all the variant MST1 constructs can be expressed and are secreted (FIG. 14).

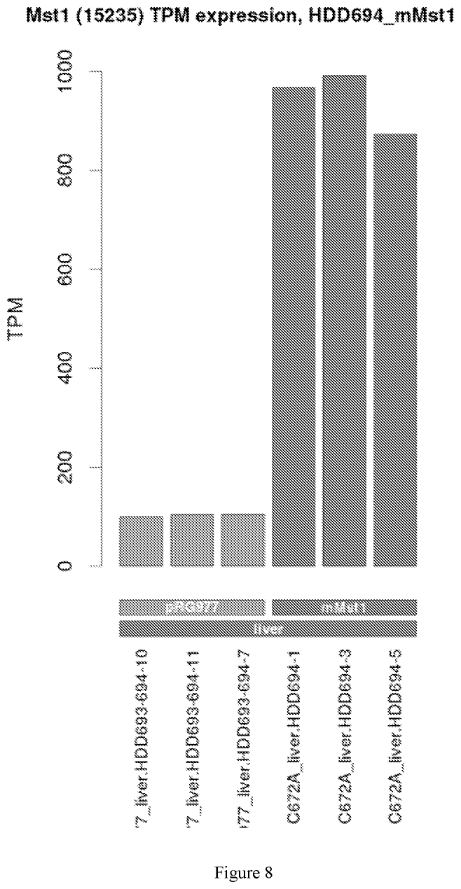

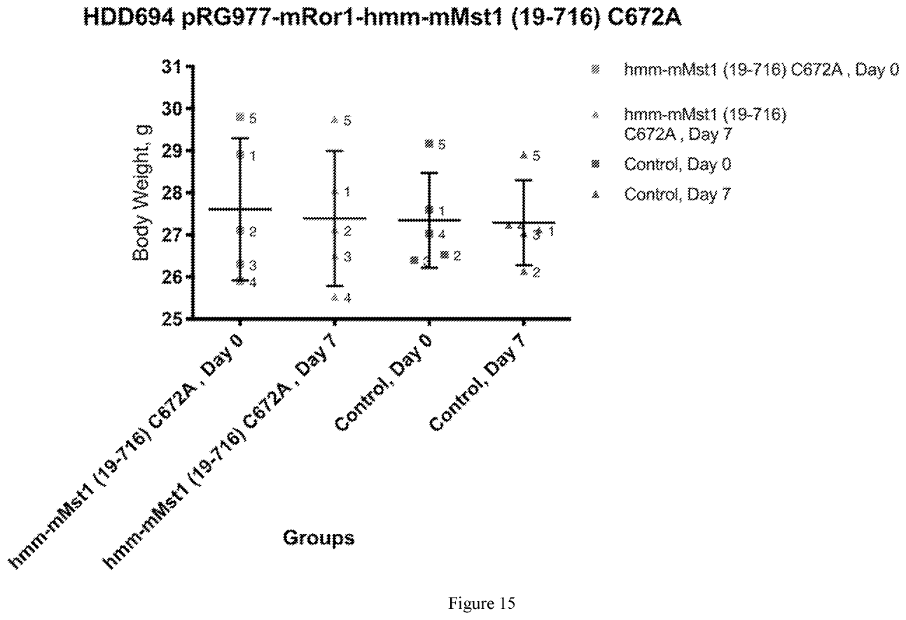

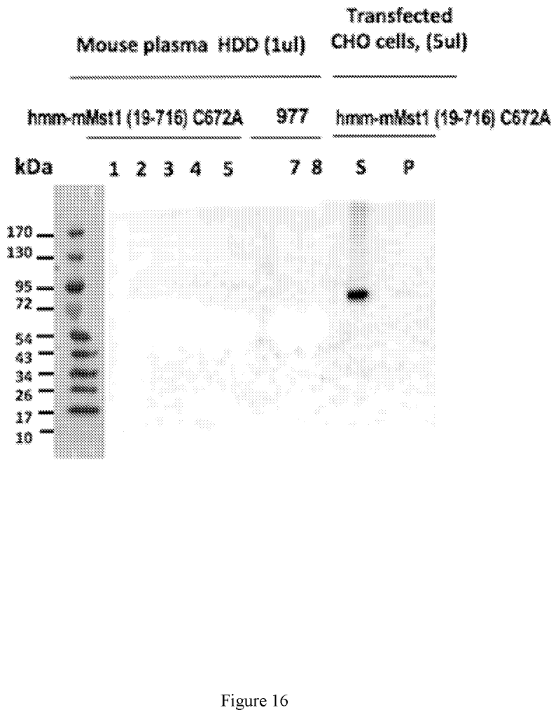

Hydrodynamic delivery (HDD) was used to deliver WT MST1 cDNA packaged in an expression construct to mice, leading to overexpression of WT MST1. Mice injected with MST1 HDD construct survived the course of experiment, indicating that MST1 overexpression is not lethal (data not shown). Consistent expression of the control HDD construct was observed in the livers of 3 mice and consistent expression of the mMst1 construct was observed in the livers of 3 mMst1-HDD mice, indicating that these constructs were expressed and that expression was consistent within each group (FIG. 8). In addition, MST1 overexpression did not appear to affect mice body weight (FIG. 15). MST1 plasma levels were below the detection level as shown in a Western blot (FIG. 16) for a protein tag that was included as part of the construct. The tag was likely cleaved and was not detected. The vector was detectable when expressed in a CHO cell line.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a patient having inflammatory bowel disease (IBD) or primary sclerosing cholangitis (PSC), the method comprising administering to the patient an agonist of the Macrophage Stimulating 1 (MST1)/Macrophage Stimulating 1 Receptor (MST1R) pathway, thereby treating the patient,
wherein the agonist of the MST1/MST1R pathway comprises:
i) a recombinant Macrophage Scavenger Receptor 1 (MSR1), recombinant Hepatocyte Growth Factor-Like protein (HGFL), or recombinant androgen receptor (AR);
ii) an antibody to MST1R;
iii) chelerythrine, recombinant Tumor Necrosis Factor Receptor Associated Factor 2 (TRAF2), or curcumin;
iv) Hepatocyte Growth Factor Activator (HGFA), matriptase, hepsin, TMPRSS11D (Human Airway Prypsin-like protease; HAT), clotting factor XIIa, clotting factor Xia, or kallikrein;

2-9. (canceled)

10. The method according to claim 1, wherein the antibody to MST1R comprises mAb Zt/g4, mAb Zt/cl, mAb Zt/f2, mAb Zt/64, mAb 3F12, mAb B9, and mAb 1G4.

11. The method according to claim 1, further comprising detecting the presence or absence of an MST1 and/or MST1R variant nucleic acid molecule or variant polypeptide associated with an increased risk of developing IBD and/or PSC in a biological sample from the patient.

12. The method according to claim 11, wherein the MST1 variant nucleic acid molecule associated with an increased risk of developing IBD and/or PSC encodes Arg703Cys or Arg651 STOP.

13. The method according to claim 11, wherein the MST1R variant nucleic acid molecule associated with an increased risk of developing IBD and/or PSC is 3:49903264:CG:C, 3:49903084:C:T, 3:49890026:G:T, 3:49902417:A:G, 3:49902560:A:T, and/or 3:49903387:G:T, or an mRNA molecule produced therefrom, or a cDNA molecule produced from the mRNA molecule.

14. The method according to claim 11, wherein detecting the presence or absence of the MST1 and/or MST1R variant nucleic acid molecule or variant polypeptide associated with an increased risk of developing IBD and/or PSC comprises:
determining whether the patient has an MST1 and/or MST1R variant genomic nucleic acid molecule associated with an increased risk of developing IBD and/or PSC, an MST1 and/or MST1R variant mRNA molecule associated with an increased risk of developing IBD and/or PSC, an MST1 and/or MST1R variant cDNA molecule produced from the mRNA molecule, and/or an MST1 and/or MST1R variant polypeptide associated with an increased risk of developing IBD and/or PSC, by:
obtaining or having obtained a biological sample from the patient; and
performing or having performed an assay on the biological sample to determine whether the patient has an MST1 and/or MST1R variant nucleic acid molecule or variant polypeptide associated with an increased risk of developing IBD and/or PSC.

15. The method according to claim 14, further comprising determining the patient's aggregate burden of having: MST1 and/or MST1R variant genomic nucleic acid molecules associated with an increased risk of developing IBD and/or PSC, MST1 and/or MST1R variant mRNA molecules associated with an increased risk of developing IBD and/or PSC, MST1 and/or MST1R variant cDNA molecules produced from the mRNA molecules, and/or MST1 and/or MST1R variant polypeptides associated with an increased risk of developing IBD and/or PSC.

16-18. (canceled)

19. The method according to claim 11, wherein the detecting step comprises sequencing at least a portion of the nucleotide sequence of the MST1 and/or MST1R nucleic acid molecule in the biological sample, wherein the sequenced portion comprises a position corresponding to a predicted loss-of-function variant position, wherein when a variant nucleotide at the predicted loss-of-function variant position is detected, the MST1 and/or MST1R nucleic acid molecule in the biological sample is an MST1 and/or MST1R predicted loss-of-function variant nucleic acid molecule.

20. The method according to claim 19, wherein the detecting step comprises:
a) contacting the biological sample with a primer hybridizing to a portion of the nucleotide sequence of the MST1 and/or MST1R nucleic acid molecule that is proximate to a predicted loss-of-function variant position;
b) extending the primer at least through the predicted loss-of-function variant position; and
c) determining whether the extension product of the primer comprises a variant nucleotide at the predicted loss-of-function variant position.

21. The method according to claim 19, wherein the detecting step comprises sequencing the entire nucleic acid molecule.

22. The method according to claim 11, wherein the detecting step comprises sequencing at least a portion of the nucleotide sequence of the MST1 and/or MST1R nucleic acid molecule in the biological sample, wherein the sequenced portion comprises a position corresponding to a missense variant position, wherein when a variant nucleotide at the missense variant position is detected, the MST1 and/or MST1R nucleic acid molecule in the biological sample is an MST1 and/or MST1R missense variant nucleic acid molecule.

23. The method according to claim 22, wherein the detecting step comprises:
a) contacting the biological sample with a primer hybridizing to a portion of the nucleotide sequence of the MST1 and/or MST1R nucleic acid molecule that is proximate to a missense variant position;
b) extending the primer at least through the missense variant position; and
c) determining whether the extension product of the primer comprises a variant nucleotide at the missense variant position.

24. The method according to claim 22, wherein the detecting step comprises sequencing the entire nucleic acid molecule.

25. The method according to claim 11, wherein the detecting step comprises:
a) amplifying at least a portion of the MST1 and/or MST1R nucleic acid molecule, wherein the portion comprises a predicted loss-of-function variant position;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the predicted loss-of-function variant position; and
d) detecting the detectable label.

26. The method according to claim 25, wherein the nucleic acid molecule in the sample is mRNA and the mRNA is reverse-transcribed into a cDNA prior to the amplifying step.

27. The method according to claim 25, wherein the detecting step comprises:
contacting the nucleic acid molecule in the biological sample with an alteration-specific probe comprising a detectable label, wherein the alteration-specific probe comprises a nucleotide sequence which hybridizes under stringent conditions to a predicted loss-of-function variant position; and
detecting the detectable label.

28. The method according to claim 11, wherein the detecting step comprises:
a) amplifying at least a portion of the MST1 and/or MST1R nucleic acid molecule, wherein the portion comprises a missense variant position;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the missense variant position; and
d) detecting the detectable label.

29. The method according to claim 28, wherein the nucleic acid molecule in the sample is mRNA and the mRNA is reverse-transcribed into a cDNA prior to the amplifying step.

30. The method according to claim 28, wherein the detecting step comprises:
contacting the nucleic acid molecule in the biological sample with an alteration-specific probe comprising a detectable label, wherein the alteration-specific probe comprises a nucleotide sequence which hybridizes under stringent conditions to a missense variant position; and
detecting the detectable label.

31. The method according to claim 11, wherein the MST1 variant nucleic acid molecule associated with an increased risk of developing IBD and/or PSC encodes Arg651STOP.


SEQUENCE LISTING (40 sequences)

SEQ ID NO: 1 - DNA, 16636 bp, Homo sapiens
  source 1..16636: mol_type = genomic DNA
ggatcctctagggtcccagctcgcctcgatggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccggtaagctgaaagaaggggcccttacttgtgagctgcatgcacatggctgaacacaggctgcttcccttccatcactgaggggatctgggggcgcggtgggggggcaggcggaaagaagcattctctctcacccagttcctaagtgctggggtggtgtgagaaaccagccagacaaaggctacactggaggcttagctcccaggctggcccttgtggctgcctcatcttgctctttcccctacctgcccatcatggcctctggttttttaggtagtttccctggctatgtgaggatagggttacataaccgtgtctctgttagcatagctcctgaatgctgggtagaatgatgggcattccctcctacttgtccaccacccatatcctcagggacacacgcagaaaaatagcactcccccctcccctcccccacccccgccaccaccaccaccaccacgcacaaatacacagggagatgctcacagattcacagactcacagctgcttgccaacagtttcactttcccttgttttgctctgctttccaaagggggtgggacgggagttgtcagagaatctccccttcctccacaccccctccccccacccacccccatccttctgccagccaccacgttccagggagcctcctgggccttggaagttgggagcttgttagggagcctgcatgtcctgggcttcactaacgtttttgccatgattgggggtgattgggggagtacccagaactcatggaatggcagcttggttctagaaggttctgggagcctcactctagtcttgtgcaagcaactataaacatctggggataagtaaaggcctaggaagggctggttggaattggtccatcccagacttttttttttaactgtatttggagccctaccccaggcaggaagtctcaaagcctgtgctgaactgggggctccttccacagactctatctaggttctctccagccatcgtgtaggcaacagcagtaagattggaccaaggaatgggacccacagctactgttcttagattctctagctatcatcctgtggccctgggcctgctgtgacattctcgaaagactcctttccgctcctattgatgcctcggagatgggagaacttacacccaatgtgaggttgggggtctttagcctccctccctctgctgagtacatctgggttgagaggccctagggttggggactccttgtctgggcctgggcaggcctgcctggccccttctccttcccaatggggagtcagaacctctccccagcttgcctagtggtcagtgggtcagaagacacccagcacggctgggcagtgccaggctccaggaagactaatcttggtccctgctcacaggctggccaggcccccaggaggtgtgtgaatcaccccgtgcagtgtttgcccagcaactctgggcatgcagccactagggcagcacccacatagtggttggcctcaggaggcctgagggactcagagctggtgcctgtcctccatgggacattggacacttacgtgttttcaggagctgggggcccctacctaagcctgggaacgtagagctaaggtcctggggctagggacaagcaggctagggtgtgggcctgactaaggtagggctctattccccttcagagccccacctgtccctgagtcatgctagaaacacctggcagccttccggctcgggccaagccaagccgatttcctgcctcaagtgaggacaaaatcccagtccccacccaccccaagctgtaggcacaaggtgtatggggtttcgaggctcagctccacatctacttttgcctccagggctactaatcagtgctgaggtctcaggactgggttaagcctctgtggtctgatggagccttcagaggctcctgcttcgaggagtgccctccgggctggtgagagagagtcctaagcacctagggctggctactgagccccgtctgcacctcgcactcagctccaacaccgcgagttcataccctggtgtactggtttcccagagcttccacagtcctcccctggagcgtgattgcagcccccaaaaacacccccttgctaaaagcaggggagaggttagtaccccctagcttccagaacttagttctggcacccaggctgtaggggtttggtttatcccttttgtaccctgaaggggggtataggaagcagccaggagtacaggcagacctttggagtctagggagaaggaccatgtccccaaccttgtgctggccaattaactccagcattcagccccttatgcaagtggggaaaccgaagcccagaggagacagggtttgctgaagtgcacacagagagtcagagaagagccagcagggggcaggagaagactcttgcggagctgagacttccaactgggctctcctcttgagggtctatgatgccttggaccagagaccaaacctcttgagcctgtttcctcatctgtgagatgtacggggtgggccaggcaccgtggctcatgcctgtaatcccagcactctgggagaccaaggcggactaatcacaaggtcaggagttcgagaccagcctggccaacatcgtgaaacccccatctctactaaaaatacaaaaaattagccaggggtggtggcgagcacctgtaatcccagctactcgggaggctgaggcaggagaatctcttgaacccgggaggtgttgcagtgagctgagatcacaccattgccctccagccccagcaacagtgtgagactccgtctcaaaaaaaaaaaaaaaaaaaaaaaaaaaaaaaaagatgtacggggtggttgtgaggatggaatgggggtaactcaccatcaggatgggcatggcagggtggggcagggagaatgaggagtggaccttccagcctgggccttctccctgcccttgctacactgagagccctgtgggggcaatgtgggatggtaaccggcaggcccagggccttctccagtcaggccaggtgaggggtgagagtgttgggctgctgtggccactgacaaaggccccagagggtcggggcacaaggctgaaccctgactccttcccttccctgacctttcccagcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtgggtcctcatccccacagtcccctagccctgggtccttgtctccatccccattttgctcacatctgacctgtcctaggtggagctggtcaggtcactaaactacttgctgtatgtgtccaacttctcactgggtgacagtgggcagcccgtgcagcgggatgtcagtcgtcttggggaccacctactctttgcctctggggaccaggtgaggtgggcaggggcagggcctggggccagggttgtgggatcacagactctccatccaatccagggaggcattgacatacaggccctaccaccctagcctactgtgtaccgggagggtctatagggcccaatctctccctccaggcacccctgagtcacctgtctttcacccacaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgaggtatggcttccctggcagggcacaggtaagagtgggacaggctgggcctggggtggtggtcagcaagtcttgatcacaatttttctcaggtctggggggtgtccttcatatctacaaggagggcattcttgtcagaggggatgccacatgcaaagacttggaggcaggaaagtgcttgaatgcaagtgactccaaatggctggagcgtggggaaggcaaggggtatggcaggagactttggggaagcaggtggggctgagagagcccaaccccacatgaggactcaggctgtttcccccatttcagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcaggtacaatctggtccctcccctccctttccctgaagggggaaaccaagcagccccttccccatgagaccctgttctctgcttatcagaggcaaggggggatgggggaagctgcagtggttctgactgctttttgagagtcaaaaaggtctctgtccctttttgagctttcatgtgcccttcctgtctgttcactcatggaccagccaaggttaatctctgccccaccagaaccttccttccatggagggaggcatgggtggagaaatgccattctctggctcagaggagccctgtggcttgtggcaggcatgcatctaggcctgtgtaattcctggctgacctcaggggttcccctggtgccccagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggtgaggctaccttgccctgtctgtgcccttggccagtgcatggtacgggaagggaggggcttggagtggaggacctgcctaagccacctctatgtcctcttaggagccagtgctgatagcagtgcaacccctctttggcccacgggcaggaggcacctgtctcactcttgaaggccagagtctgtctgtaggcaccagccgggctgtgctggtcaatgggactgagtgtctgctagcacggtaagtaccaccaggcaggcatgggaggccgcagggttctatccaggtggggacccggattctgtcaggatggagggaagagtgcttgttacatggtcagtgtggtgtggagagcaggtccaggaagaaaagactactgccctcttcagcccagaggcaagagagcccatcacagagtccaatggctccagccaaggctgtggggaacctgttccctccttaccctcttgggatgtcttcctgggaccaccttattttcagacagaccatggagtggggcaggtgggagagataacagacgaactgggagaggtcaacaggcccctggaagaagtgggcctggccatcacaaagagggtaaccttgagcccaatctcttgttcctgtagggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatgtaagcactgcctctttgcccactctggcctctggggaatgagagagccagctttggagaacacccaaagcctaccaccctaccctcttccacagcaactcccacatcaccatctgtggccagcatctaacttcagcatggcacttagtgctgtcattccatgacgggcttagggcagtggaaagcagggtgagtgagtgctggccaggagggaggaaggctggatgagttccctgagctgcagcccaacctcgtgctgggctgaggcgaggagcaatcacaggtggggttcctggctaatcactctcatattggtcccagcagtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtaagtgtaagggataggggcagggacagttggggatctgaaagtaggggccagcctactggctggtcctcatgaccctctctgcagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtaggcagcccagctggacctccctgggaaacacgggcagagggcctacaggctgggcctgagttgccacctgcccccaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctaggtgagttctctgctcctacctttaatcagcccctacccccaaccaatggcatcttcaagtccctacatcctctctctgcccaggacttagggatctaagtcctccccagaggggtcggcccctaggagcttggaaagccaagccaccaaggattctctttccacagttcttcctcccaacctgaatgacctggcatccctggaccagactgctggagccacacccctgcctattctgtactcgggctctgactacagaagtggccttggtgagatagtggaggcatgagaagctaaagccaccccctgtccctacaagctcctcattccctctccccacagcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggtgtgggggccaggtggggctggggcagagatggagtctcaagatgacataggctaggccaggcgtggtggctcactcctgtaatcccagcactttgggaggccgaggcgggcagatcatgaggtcaggagatcgagaccgtcctggctaacacagtgaaaccccgtctctactaaaaatacaaaaaaatattagccagccatggtggcaggcgcctgtagtcccagctacttgggaggctgaggcaggagaatggcatgaactcgggaggcggagcttgcagtgagccaagattgcaccactgcactccagcctgggcgacagagcgagactccttctcaaaaaaaaaaaaaatgacatgggctaaagggacagttggaacagatactgctctaaccttctcaccaacctgtgtgaccttgggtataccacttaacctctctgagcctccatttgctcatctgtaaaatggggatgagaaaagttctgtcttcagtgggttctggtgtggatcaatcaggcataggaagattgtagcctctgccatcaagtggctgctgtttgtttttggttttatttatggttttgtttttgtttttgtgacagggtctcactctgttacccaggctggagtgcaatggcacaatcatggctcactgcagcctcgacctccccagcctcaggtgatcctcctgcctcagcctcctgagtagctgggattacaatcatgcaccaccatgcctggctaattttttgtttattttttgtagagatgggcctcgaatcatgttgtccaggctggcctcgaactcctggactcaagcaatctgcctgcctaaccttcccaaagtgctgggattacaggcatgagccaccgaacccagcctgttttattatatttattttattttatttttatttttattctattttattattattattttttttgagacagaatttcactcttgttgcccaggctggagtgcaatggcgcgatctcggctcattgcaacctcctcctcccgggttcaagtgattctcctgtctcagcttcctgagtagctaggattacaggcgcctgccacaacacccggctaatttttagtatttttggtagagataggatttcaccatgttggccaggccggtctcgaactcctgacctcaggtgatccgcccgcctcagcctcccaaagtgctaggattacaggcatgagccactgcccccagcctattttattttattttattttttttttttttttgagagggagtctcactctttcacccaggctggagtgcagtggcgcgatctcgactcactgcaagctccgcctcccaggttcatgccattctcctgcctcagcctcccaagtagctgggactacaggcgcctgccaccgtgcccggctaattttttgtatttttagtagagatggggtttcaccgtgttagccaggatggtctcgatctcctgacctcatgatccacccgcctcagcctcccaaagtgctgggattacaggtgtgagccaccgcgcccggcctattttattttttcaagatggagtcttgctctatcacccaggctggagtgcagtggtgcaatcttggctcactgcaacctccccctcccaggttcaagcaattctcctgcctcaccctccctagtagctgggattacaggcgcccgccaccacacctggctaatttttgtatttttagtagagatggggtttcaccatgttggccaggctggtctcaaactcctaacctcaggtgatctgcctgccttgatctcccaaagtgctgggattacaggcgtgagccactgtatccagcctatttatttatttatttatttatttatttatttatttatttttgagacagaacctcactctgtcacccaggctggagtgcagtggcatgatctcagctcactgcaacctctgcctcccggattcaagtgattctcctgcctcagcctcctaagtagctgggattacagacgtgcaccaccaagcccgtctaatttttgtatttttagtagaaatggagtttcatcatgttggccaggctggtctcaaactcccgacctcaggtgatccgcccgcctcggcctcccgaagtgctgggattacaggcgtgagccatcgcacccgaccctgctttatttacttatttatttatttttatttttttgagacagagtctcactctattgcccaggctggagtgcagtagtgcaatctcggctcactgcaacctctgcctcctggattcaagcatttctcctgcctcagcctcctgagtagctgggattacaggggcctgccactgccaccacacctgtctaatttttgtagttttagtagagacggggtttcatcatgttggccagactggtcttgaacccctgacctcagctgatcctcccgccccagcatcccaaagtattaggattaaaggcatgagccttttttttttttttttaatttaaaaaaaagacagcatctcactatgttacccaggctggccttgaactcctgggcttatgcgattctcttgcctcagcctcctgagtaactgggattacaggcgcactgcgcttggcttctgctgcttttctttgagggggatggagtctcgccctgtctctcaggctggagtgcagtgacacgatcttggctcactgcaacctctgcctcccgggttcaagctatcctcctgcctcaggctcccaagcagctgggactagagatggggtttcaccatattagccaggctggtctcaaactcctgacctcgtgatccacccgccttggcctctcaaagtgctggcattacaggtgtgagccactgcacgcagccctgctgcttttttaaaatgtcagattgggccgggtgtggtggctcacacctgtaatcccagcactttgggaggcctaggtgggtgaatcacaagatcaggagtttgagaccagcctggccaacatggtgaaacccagtctctactaaaaatacaaaaaaattagctgtgtgtagtggcagttgcctgtaatcccagctactcaggaggctgaggcaggagaatcacttgaacctgggaggcagaggttgcaatgagttgagattgcaccactgcacttcagcctgggcaagagtgagactcggtctcaaaaaaaataaataaaaattaaaaataaataaaatgtcagattggccagtttagtgctctgaggtctggttgccaatgagaatgtgtggtgtgagccttgggccctggatcccctctcatcgactcattatgcacctcacaccaggccactttggagttgtctaccacggagaatacatagaccaggcccagaatcgaatccaatgtgccatcaagtcactaagtcgtaagtggggcagaagatgggaaggcagagggaggggcctcaggctgggaggattctttttctctgtgttcccaccttactgagtgaccctgagcaggagaccttcatttcccatttccccatctgagccttataaagggacgccctgccctgtgcctgacttgcgctgctctgcaggcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcgggtcagtgttcatctggctctgggttgggggctgggcagcagctggagaaggagctgctgtgcccttgcccaccaacccacctgtgcccccagaaccccaccgtgaaggacctcatcagctttggcctgcaggtagcccgcggcatggagtacctggcagagcagaagtttgtgcacagggacctggctgcgcggaactgcatgtgagagtccagagtagctggggtgaagcaaaaggacagggcatgagggtgtgcggtgctcaaggccgcctcagggaagggcccactccagccttgtcctcccctctctgcaccttactttttttgtctgcccagtgcagacttggactggacactgtggctttgaaatttggccaggcgcggtggctcacgtctgtaatcccagcactttgggaggctgaggaaggcggatcatgaggtcaggagatcgagaccatcctggctaacacggtgaaaccccgtctctactaaaaatacaaaaagattagccaggcgtggtggcgggcccctgtagtcccagctactcaggaggctgaggcgggagaatggcgtgaccccaggaggcagagcttgcagtgagcggagatcgcgccactgcactccagcctgggtgacagagcgagactccgtctcaaaaaaaaaaaaaattctgtaagggcctagaacaggtgggttcagggcctggtaagggccagtcctaagtgtgatcctctccctacccctaaggctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtggtgaggccccacctgccctacagtccacatggctttggcacccaagaacactgggcctcacccattcagctctgaatgatctgagcccagggagtccccagggtagatgacccccacagcccatctgctcacctcttgggcagatgtaagatggaatctgacacctgccagggctagaggaggctgggaactcactggctcctcacagataatccagggcctgcccagccccaactctgggtaggatgaggtagtgggaggagacagaggaaagacacaggtgatgtattgaggaatgagctctcagtggagggtagggtcttccatttccagctgaaggactctgggaggtagaagccctgggggacccacctgcccccaaatttggggtgagttgagtcccctcacctccccctttccacagtggtcatttggtgtgctgctgtgggaactgctgacacggggtgccccaccataccgccacattgacccttttgaccttacccacttcctggcccagggtcggcgcctgccccagcctgagtattgccctgattctctgtgagtatgtggaggtggtagtggggagggaactgggccccagagacatggaagagacaggagacagagctcctgcctggcttcctgactgaacttctggttgactaggaaagggactctcgctgggcctcaatttcctcatctgtgaactggtcccaccctcatgcagagtaggaccctgggaggacctcacagttgccatggtaacagcattctgagctcagagttggggttgaggcccgtggtttgattttcccagtgctcctcctacccgactcccactaacggggtctgctttccaccttttcctctcatttaatctcgaggaaagattccataacccccacagcaaccccctctggatctccaggtcaggttttttcactgtatgtggtttcagattccccattcctggagggatggcggcagctgcattgtcctcagtgcctacggggtgggttcggtgctgaggttcagcttcatacgtagcaggtgtgcctgagctgtggctaggtctgctgggttagaacagagagaaccaggacataggtaaaagtagttaacgcctcctgtctcaggctgtgctccaagaaggatgcgagtagcccttcctcacagtcctacctgccttgggtttgctcttactattcacttctttaattcactgcctacccatatccagattgggctcctggaaacagactcagctggagatttatgtacggggatttactgagaggttgggggcaagggaagaaggactgggcaggagaaatgtcacagaaactgcagcccatcccacagggactgcttgaactgggatgacccaccacagttttctgaaattgagaccaacaggccaggtctttgtaccaggcattaatcagttactagtcacaggctgtccctggtgaggggatgtgagcttggcagaggcagttctcttctgcctagggcaattcctagagagagcctcagtcaagaaccatcagcaggcaacactcctagccactgggaaagtaaaggggtatctgggtggcacaccatagggtccacttcagcagtccagcccttctcctctctgggcctcaatttcctcatctgtaaagtggagacaacagtaccaccttccgagagtctgagaattatatgagatattgtgtgtagggtagagtattatttcaggcaggaatgacatccccaagataatggggcaggcactaggacccagcaggtaaacctggggaacctctgcttcaggaccagggtgccactggtgctgccccaaatgctcttgcttcagaatttttttttttttttttgagacagagtctcactctgatgcccaggctggagtgcggtggcgcaatgttggctcactgcaacctctgcctcccaggttcaagtgattctcctacctcagcctcctgagtaactgggactacaggcgtgtgccaccacgcccagtaattttttttttttttttttgagatggagtctcgctctgttgcccaggctggagtgcagtggtgcaatctcggctcactgcaagctccgcctcccgggttcacgccattctcctgcctcagcctcccgagtagctgggactacaggcgcctgccacgacgcccggctaatattttgtgtttttagtagagacggggtttcactgtgttagccaggatggtctcgatctcctgacctcgtgatccacccaccttggcctcccaaagtgctgggattacaggcgtgagccaccacgcccagctttgcccagttaatttttgtatttttagtagagatggggtttcactatgttggccaggctggtctcgaactcctgacctcgtgacctgcccacctcagcctcccaaagtgctgggattacaggcatgagccactgcccccggccacttcaggatttttaattcctgctttgctggacccaggcttccttcctggaagaactgtgatgatgaccattgtacctcgtgagctgggccttgtgcctagcacttcacaggcatcagcctatatgcctacttctctgtgatcagaggaggacccagaggcacagagaagttaaggaacttatccaagatcacatagcccaggccacagggcacaggagcccaagctcttaacacctgaggtagttcgacctctgccagggtgggaaggcaggactggaacagtaatcatagggatgagaagagctgcccctgattgggtagtgctaggtactaggtcctgtcatctatcctcatccctgtgtggcctgccagaggtttaacagcaagtgggtggcccagaatccttgggtggaaattgccttaacctgacatccctttcccccaacaggtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttgacttagttcttgggctggacctgcttagctgccttgagctaaccccaagctgcctctgggccatgccaggccagagggcagtggccctccaccttgttcctgccctttaactttcagaggcaataggtaaatggggcccattaggtccctcactccacagagtgagccagtgagggcagtcctgcaacatgtatttatggagtgcctgctgtggaccctgtcttctgggcacagtggactcagcagtgaccacaccaacactgacccttgaaccaataaaggaacaaatgactattaaagcacaaa

SEQ ID NO: 2 - DNA, 4221 bp, Homo sapiens
  source 1..4221: mol_type = genomic DNA
ggatcctctagggtcccagctcgcctcgatggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcaggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttgacttagttcttgggctggacctgcttagctgccttgagctaaccccaaggctgcctctgggccatgccaggccagagcagtggccctccacttgttcctgccctttaactttcagaggcaataggtaaatgggcccattaggtccctcactccacagagtgagccagtgagggcagtcctgcaacatgtatttatggagtgcctgctgtgaccctgtcttctgggcacagtggactcagcagtgaccacaccaacactgacccttgaaccaataaaggaacaaatgactattaaagcacaaaaaaaaaa

SEQ ID NO: 3 - RNA, 4541 nt, Homo sapiens
  source 1..4541: mol_type = mRNA
ggatcctctagggtcccagctcgcctcgatggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcaggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccgaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttgacttagttcttgggctggacctgcttagctgccttgagctaaccccaaggctgcctctgggccatgccaggccagagcagtggccctccaccttgttcctgccctttaactttcagaggcaataggtaaatgggcccattaggtccctcactccacagagtgagccagtgagggcagtcctgcaacatgtatttatggagtgcctgctgtggaccctgtcttctgggcacagtggactcagcagtgaccacaccaacactgacccttgaaccaataaaggaacaaatgactattaaagcacaaaaaaaaaa

SEQ ID NO: 4 - RNA, 4139 nt, Homo sapiens
  source 1..4139: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 5 - RNA, 765 nt, Homo sapiens
  source 1..765: mol_type = mRNA
atggagctcctcccgccgctgcctcagtccttcctgttgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 6 - RNA, 1725 nt, Homo sapiens
  source 1..1725: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 7 - RNA, 2727 nt, Homo sapiens
  source 1..2727: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggcagtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtaagtgtaagggataggggcagggacagttggggatctgaaagtaggggccagcctactggctggtcctcatga

SEQ ID NO: 8 - RNA, 1626 nt, Homo sapiens
  source 1..1626: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcaggtacaatctggtccctcccctccctttccctgaagggggaaaccaagcagccccttccccatga

SEQ ID NO: 9 - RNA, 1944 nt, Homo sapiens
  source 1..1944: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcaggtacaatctggtccctcccctccctttccctgaagggggaaaccaagcagccccttccccatga

SEQ ID NO: 10 - RNA, 1488 nt, Homo sapiens
  source 1..1488: mol_type = mRNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtgggtcctcatccccacagtcccctagccctgggtccttgtctccatccccattttgctcacatctga

SEQ ID NO: 11 - DNA, 4221 bp, Homo sapiens; identical to SEQ ID NO: 2
  source 1..4221: mol_type = genomic DNA

SEQ ID NO: 12 - DNA, 4541 bp, Homo sapiens
  source 1..4541: mol_type = genomic DNA
ggatcctctagggtcccagctcgcctcgatggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcaggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccgaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttgacttagttcttgggctggacctgcttagctgccttgagctaaccccaaggctgcctctgggccatgccaggccagagcagtggccctccaccttgttcctgccctttaactttcagaggcaataggtaaatgggcccattaggtccctcactccacagagtgagccagtgagggcagtcctgcaacatgtatttatggagtgcctgctgtggaccctgtcttctgggcacagtggactcagcagtgaccacaccaacactgacccttgaaccaataaaggaacaaatgactattaaagcacaaaaaaaaaa

SEQ ID NO: 13 - DNA, 4139 bp, Homo sapiens
  source 1..4139: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgagtatattgggctgggcgctgtggctgactgtgtgggtatcaacgtgaccgtgggtggtgagagctgccagcacgagttccggggggacatggttgtctgccccctgcccccatccctgcagcttggccaggatggtgccccattgcaggtctgcgtagatggtgaatgtcatatcctgggtagagtggtgcggccagggccagatggggtcccacagagcacgctccttggtatcctgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 14 - DNA, 765 bp, Homo sapiens
  source 1..765: mol_type = genomic DNA
atggagctcctcccgccgctgcctcagtccttcctgttgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgctggacgagtcattcacagtcaaggtggctgactttggtttggcccgcgacatcctggacagggagtactatagtgttcaacagcatcgccacgctcgcctacctgtgaagtggatggcgctggagagcctgcagacctatagatttaccaccaagtctgatgtgtggtcatttggtgtgctgctgtgggaactgctgacacggggtgccccaccataccgccacattgacccttttgaccttacccacttcctggcccagggtcggcgcctgccccagcctgagtattgccctgattctctgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 15 - DNA, 1725 bp, Homo sapiens
  source 1..1725: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctggcagcttcagcccacgctcagtgtctatcaggcgtctcaaggctgacgcctcgggattcgcaccgggctttgtggcgttgtcagtgctgcctttgctgctgcttgtggctgcactggcgactgcactggtcttcagctactggtggcggaggaagcagctagttcttcctcccaacctgaatgacctggcatccctggaccagactgctggagccacacccctgcctattctgtactcgggctctgactacagaagtggccttgcactccctgccattgatggtctggattccaccacttgtgtccatggagcatccttctccgatagtgaagatgaatcctgtgtgccactgctgcggaaagagtccatccagctaagggacctggactctgcgctcttggctgaggtcaaggatgtgctgattccccatgagcgggtggtcacccacagtgaccgagtcattggcaaaggccactttggagttgtctaccacggagaatacatagaccaggcccagaatcgaatccaatgtgccatcaagtcactaagtcgcatcacagagatgcagcaggtggaggccttcctgcgagaggggctgctcatgcgtggcctgaaccacccgaatgtgctggctctcattggtatcatgttgccacctgagggcctgccccatgtgctgctgccctatatgtgccacggtgacctgctccagttcatccgctcacctcagcggaaccccaccgtgaaggacctcatcagctttggcctgcaggtagcccgcggcatggagtacctggcagagcagaagtttgtgcacagggacctggctgcgcggaactgcatgtaccaagtgatgcagcaatgctgggaggcagacccagcagtgcgacccaccttcagagtactagtgggggaggtggagcagatagtgtctgcactgcttggggaccattatgtgcagctgccagcaacctacatgaacttgggccccagcacctcgcatgagatgaatgtgcgtccagaacagccgcagttctcacccatgccagggaatgtacgccggccccggccactctcagagcctcctcggcccacttga

SEQ ID NO: 16 - DNA, 2727 bp, Homo sapiens
  source 1..2727: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcagaccagtgccccggaaagactttgtagaggagtttgagtgtgaactggagcccttgggcacccaggcagtggggcctaccaacgtcagcctcaccgtgactaacatgccaccgggcaagcacttccgggtagacggcacctccgtgctgagaggcttctctttcatggagccagtgctgatagcagtgcaacccctctttggcccacgggcaggaggcacctgtctcactcttgaaggccagagtctgtctgtaggcaccagccgggctgtgctggtcaatgggactgagtgtctgctagcacgggtcagtgaggggcagcttttatgtgccacaccccctggggccacggtggccagtgtcccccttagcctgcaggtggggggtgcccaggtacctggttcctggaccttccagtacagagaagaccctgtcgtgctaagcatcagccccaactgtggctacatcaactcccacatcaccatctgtggccagcatctaacttcagcatggcacttagtgctgtcattccatgacgggcttagggcagtggaaagcaggcagtgtgagaggcagcttccagagcagcagctgtgccgccttcctgaatatgtggtccgagacccccagggatgggtggcagggaatctgagtgcccgaggggatggagctgctggctttacactgcctggctttcgcttcctacccccaccccatccacccagtgccaacctagttccactgaagcctgaggagcatgccattaagtttgaggtaagtgtaagggataggggcagggacagttggggatctgaaagtaggggccagcctactggctggtcctcatga

SEQ ID NO: 17 - DNA, 1626 bp, Homo sapiens
  source 1..1626: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcaggtacaatctggtccctcccctccctttccctgaagggggaaaccaagcagccccttccccatga

SEQ ID NO: 18 - DNA, 1944 bp, Homo sapiens
  source 1..1944: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtggagctggtcaggtcactaaactacttgctgtatgtgtccaacttctcactgggtgacagtgggcagcccgtgcagcgggatgtcagtcgtcttggggaccacctactctttgcctctggggaccaggttttccaggtacctatccaaggccctggctgccgccacttcctgacctgtgggcgttgcctaagggcatggcatttcatgggctgtggctggtgtgggaacatgtgcggccagcagaaggagtgtcctggctcctggcaacaggaccactgcccacctaagcttactgagttccacccccacagtggacctctaaggggcagtacaaggctgaccctgtgtggctccaacttctaccttcacccttctggtctggtgcctgagggaacccatcaggtcactgtgggccaaagtccctgccggccactgcccaaggacagctcaaaactcaggtacaatctggtccctcccctccctttccctgaagggggaaaccaagcagccccttccccatga

SEQ ID NO: 19 - DNA, 1488 bp, Homo sapiens
  source 1..1488: mol_type = genomic DNA
atggagctcctcccgccgctgcctcagtccttcctgttgctgctgctgttgcctgccaagcccgcggcgggcgaggactggcagtgcccgcgcaccccctacgcggcctctcgcgactttgacgtgaagtacgtggtgcccagcttctccgccggaggcctggtacaggccatggtgacctacgagggcgacagaaatgagagtgctgtgtttgtagccatacgcaatcgcctgcatgtgcttgggcctgacctgaagtctgtccagagcctggccacgggccctgctggagaccctggctgccagacgtgtgcagcctgtggcccaggaccccacggccctcccggtgacacagacacaaaggtgctggtgctggatcccgcgctgcctgcgctggtcagttgtggctccagcctgcagggccgctgcttcctgcatgacctagagccccaagggacagccgtgcatctggcagcgccagcctgcctcttctcagcccaccataaccggcccgatgactgccccgactgtgtggccagcccattgggcacccgtgtaactgtggttgagcaaggccaggcctcctatttctacgtggcatcctcactggacgcagccgtggctgccagcttcagcccacgctcagtgtctatcaggcgtctcaaggctgacgcctcgggattcgcaccgggctttgtggcgttgtcagtgctgcccaagcatcttgtctcctacagtattgaatacgtgcacagcttccacacgggagccttcgtatacttcctgactgtacagccggccagcgtgacagatgatcctagtgccctgcacacacgcctggcacggcttagcgccactgagccagagttgggtgactatcgggagctggtcctcgactgcagatttgctccaaaacgcaggcgccggggggccccagaaggcggacagccctaccctgtgctgcgggtggcccactccgctccagtgggtgcccaacttgccactgagctgagcatcgccgagggccaggaagtactatttggggtctttgtgactggcaaggatggtggtcctggcgtgggccccaactctgtcgtctgtgccttccccattgacctgctggacacactaattgatgagggtgtggagcgctgttgtgaatccccagtccatccaggcctccggcgaggcctcgacttcttccagtcgcccagtttttgccccaacccgcctggcctggaagccctcagccccaacaccagctgccgccacttccctctgctggtcagtagcagcttctcacgtgtggacctattcaatgggctgttgggaccagtacaggtcactgcattgtatgtgacacgccttgacaacgtcacagtggcacacatgggcacaatggatgggcgtatcctgcaggtgggtcctcatccccacagtcccctagccctgggtccttgtctccatccccattttgctcacatctga

SEQ ID NO: 20 - protein, 1294 aa, Homo sapiens [sequence omitted]

SEQ ID NO: 21 - protein, 1400 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAGSFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLQVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIRGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 22 - protein, 1080 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVTLELSTTENT

SEQ ID NO: 23 - protein, 254 aa, Homo sapiens
MELLPPLPQSFLLLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 24 - protein, 574 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAGSFSPRSVSIRRLKADASGFAPGFVALSVLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 25 - protein, 908 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRQCERQLPEQQLCRLPEYVVRDPQGWVAGNLSARGDGAAGFTLPGFRFLPPPHPPSANLVPLKPEEHAIKFEVSVRDRGRDSWGSESRGQPTGWSS

SEQ ID NO: 26 - protein, 541 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRYNLVPPLPFPEGGNQAAPSP

SEQ ID NO: 27 - protein, 647 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRYNLVPPLPFPEGGNQAAPSP

SEQ ID NO: 28 - protein, 495 aa, Homo sapiens
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVGPHPHSPLALGPCLHPHFAHI

SEQ ID NO: 29 - DNA, 5107 bp, Homo sapiens
  source 1..5107: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctggtaagtgcccccaaccctgatccccatctgccttcaggagggggttggccccattctcctattctaggatgagaaaaaagtcggggagccagaggctcagtgggcatggggcagtgaccttgccctcttgagcacagctgggaagccctaggaacacatagacattgcccacttaggcctctattagcacgtctgctctagcactgaagcagtgtcaggaccacacagatgcacgcacacagcaggcagtgacccctcctgagcctgatctacccctctaacctagcatatgcctttgtgcaggtgagagcccagatttggagtctgaatgcctagccagggcccctggctgggtaatgtgatggctctgagccttagcattctcatttgagagatgaggtggggcaagcttcatcacccactgctctcacagagcgtatgtgttagatctgagcccggtgcctgggccactaaacagaggcaccggtgataactaccaagtctgggcctgcttcccaggggaaatttttttcacaagtatctgtgcagggggctagactggcccttgaaagtgcatacagggtccatcccagaagccttgtagctttgatcccctgaatgaacaaagtgtggacatgccaatacacattactgacatgtatgcccacctgacctgcacccactcatgcctactctgcagggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccggtgagtggccactgggcctagataagactgggggcagggaagcctgggctgtggcgttaccctgtgccttcttctctccagggccttccactacaacgtgagcagccatggttgccaactgctgccatggactcaacactcgccccacacgaggctgcggcgttctgggcgctgtgacctcttccagaagaaaggcaagtgggggtggagaggggcagggtgggagacaggggacctcagcccaagttgatcttctgtctcttgctcccagactacgtacggacctgcatcatgaacaatggggttgggtaccggggcaccatggccacgaccgtgggtggcctgccctgccaggcttggagccacaagttcccaaatgatcacaagtgagacaaacaccttccctccgtcccggcctggggccttcccccagcacacactatagtgatgctctgggccctcaggtacacgcccactctccggaatggcctggaagagaacttctgccgtaaccctgatggcgaccccggaggtccttggtgctacacaacagaccctgctgtgcgcttccagagctgcggcatcaaatcctgccgggagggtaagcggcgccgggtcaagctgggagagtggagggacaagcccacgtccatccacgaacccactggctctttgtctccagccgcgtgtgtctggtgcaatggcgaggaataccgcggcgcggtagaccgcacggagtcagggcgcgagtgccagcgctgggatcttcagcacccgcaccagcaccccttcgagccgggcaagtacgcgtaggcggtatcggcgtcctgggggccgggctagggaaggtccaggactccaggggcagggctccgtgtagggcaattgggcggggccagataagccagagtcccagggtcttgttcacgccccattaccgcccccaggttcctcgaccaaggtctggacgacaactattgccggaatcctgacggctccgagcggccatggtgctacactacggatccgcagatcgagcgagagttctgtgacctcccccgctgcggtaggcggcggggaccaggcctgggagggtacctgggaaccttggggaggggcgtggcttggccggggaggtaagaggggctgggcgtgacctgagagcataccccgtggagtaccgtacacctgggaaaggcgggtttggtcccagccccagagggatctcagctctcgctcggggcccgacctatctcggtccatctaagggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaagtgaggtggggggggggggcgggcgttgggacgtgctgctgcgggtgagacgggaggaaggtagtcacgggctcaaggctggaggctggcgggctagggctgagtggagcgcctgcttagagaccttcgggagaacttctgccggaaccccgacggctcagaggcgccctggtgcttcacactgcggcccggcatgcgcgcggccttttgctaccagatccggcgttgtacagacgacgtgcggccccagggtgaggcccaagcttgggggctacagagccgggctggaagcctggaaccggagggccggggcgaggtctcggcctgatggctgcccgcacccgccacagactgctaccacggcgcaggggagcagtaccgcggcacggtcagcaagacccgcaagggtgtccagtgccagcgctggtccgctgagacgccgcacaagccgcagtgagtccctggtgctcccggccccgccagggccctaaccctggggcggcatgctttggtgtctgggaccagagcctggaaatggttgagactaccctgccacgattttgctcccgcttccgcctaggttcacgtttacctccgaaccgcatgcacaactggaggagaacttctgccggaacccagatggggatagccatgggccctggtgctacacgatggacccaaggaccccattcgactactgtgccctgcgacgctgcggtgagcactagtgacgcttcccccatgaccctgcctcagcccccacccaaaggctggctcccttaaccccagtgaactttgtctttcagctgatgaccagccgccatcaatcctggaccccccaggttaggagttgggccagttatgggtcaggccctttagcccacgacatccacacagtctgggtttcatccagcccaccccatcctacagaccaggtgcagtttgagaagtgtggcaagagggtggatcggctggatcagcggcgttccaagctgcgcgtggttgggggccatccgggcaactcaccctggacagtcagcttgcggaatcggtgaggcacaactgcctgtctcccacagagaggagctgaggttgtgtcctctgtggttatcccactgggggctgggaatctatccctgcccccagaggtcctagccagaagatggcaggtctagcatctgtcccaggagtctgttacctgtcctaattccccactcctctaggcagggccagcatttctgcggggggtctctagtgaaggagcagtggatactgactgcccggcagtgcttctcctcctggtgagcctcccttgtgtttggggacccagtctcatcccaccttcccccttccccaggcaagctaacaagtgagccttggggcaatggactgagagtcacaaatgacctagcagagcttctctcccagccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagaggtatgtggacaacctgggagggtgtgaggtggggctgggccttgtggcctcagaccctgagtgcccccattcttgctaaagatctgtgaccctgaaccagcgtgtggccctgatctgcctgccccctgaatggtatgtggtgcctccagggaccaagtgtgagattgcaggctggggtgagaccaaaggtaagagcacagtgcacaggactgctggtggccaggaggcccagccctggatcttcctgcaggaccctctccctctccccattcccctcactgcaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgaggttggtggcagggccctgggccagccctggaagggtatggggggctagaaatgaactattttatcatgaagcaggctagtcattgctgtggcccggggccctcatcagttctcctacctgccagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 30 - DNA, 5107 bp, Homo sapiens
  source 1..5107: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctggtaagtgcccccaaccctgatccccatctgccttcaggagggggttggccccattctcctattctaggatgagaaaaaagtcggggagccagaggctcagtgggcatggggcagtgaccttgccctcttgagcacagctgggaagccctaggaacacatagacattgcccacttaggcctctattagcacgtctgctctagcactgaagcagtgtcaggaccacacagatgcacgcacacagcaggcagtgacccctcctgagcctgatctacccctctaacctagcatatgcctttgtgcaggtgagagcccagatttggagtctgaatgcctagccagggcccctggctgggtaatgtgatggctctgagccttagcattctcatttgagagatgaggtggggcaagcttcatcacccactgctctcacagagcgtatgtgttagatctgagcccggtgcctgggccactaaacagaggcaccggtgataactaccaagtctgggcctgcttcccaggggaaatttttttcacaagtatctgtgcagggggctagactggcccttgaaagtgcatacagggtccatcccagaagccttgtagctttgatcccctgaatgaacaaagtgtggacatgccaatacacattactgacatgtatgcccacctgacctgcacccactcatgcctactctgcagggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccggtgagtggccactgggcctagataagactgggggcagggaagcctgggctgtggcgttaccctgtgccttcttctctccagggccttccactacaacgtgagcagccatggttgccaactgctgccatggactcaacactcgccccacacgaggctgcggcgttctgggcgctgtgacctcttccagaagaaaggcaagtgggggtggagaggggcagggtgggagacaggggacctcagcccaagttgatcttctgtctcttgctcccagactacgtacggacctgcatcatgaacaatggggttgggtaccggggcaccatggccacgaccgtgggtggcctgccctgccaggcttggagccacaagttcccaaatgatcacaagtgagacaaacaccttccctccgtcccggcctggggccttcccccagcacacactatagtgatgctctgggccctcaggtacacgcccactctccggaatggcctggaagagaacttctgccgtaaccctgatggcgaccccggaggtccttggtgctacacaacagaccctgctgtgcgcttccagagctgcggcatcaaatcctgccgggagggtaagcggcgccgggtcaagctgggagagtggagggacaagcccacgtccatccacgaacccactggctctttgtctccagccgcgtgtgtctggtgcaatggcgaggaataccgcggcgcggtagaccgcacggagtcagggcgcgagtgccagcgctgggatcttcagcacccgcaccagcaccccttcgagccgggcaagtacgcgtaggcggtatcggcgtcctgggggccgggctagggaaggtccaggactccaggggcagggctccgtgtagggcaattgggcggggccagataagccagagtcccagggtcttgttcacgccccattaccgcccccaggttcctcgaccaaggtctggacgacaactattgccggaatcctgacggctccgagcggccatggtgctacactacggatccgcagatcgagcgagagttctgtgacctcccccgctgcggtaggcggcggggaccaggcctgggagggtacctgggaaccttggggaggggcgtggcttggccggggaggtaagaggggctgggcgtgacctgagagcataccccgtggagtaccgtacacctgggaaaggcgggtttggtcccagccccagagggatctcagctctcgctcggggcccgacctatctcggtccatctaagggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaagtgaggtggggggggggggcgggcgttgggacgtgctgctgcgggtgagacgggaggaaggtagtcacgggctcaaggctggaggctggcgggctagggctgagtggagcgcctgcttagagaccttcgggagaacttctgccggaaccccgacggctcagaggcgccctggtgcttcacactgcggcccggcatgcgcgcggccttttgctaccagatccggcgttgtacagacgacgtgcggccccagggtgaggcccaagcttgggggctacagagccgggctggaagcctggaaccggagggccggggcgaggtctcggcctgatggctgcccgcacccgccacagactgctaccacggcgcaggggagcagtaccgcggcacggtcagcaagacccgcaagggtgtccagtgccagcgctggtccgctgagacgccgcacaagccgcagtgagtccctggtgctcccggccccgccagggccctaaccctggggcggcatgctttggtgtctgggaccagagcctggaaatggttgagactaccctgccacgattttgctcccgcttccgcctaggttcacgtttacctccgaaccgcatgcacaactggaggagaacttctgccggaacccagatggggatagccatgggccctggtgctacacgatggacccaaggaccccattcgactactgtgccctgcgacgctgcggtgagcactagtgacgcttcccccatgaccctgcctcagcccccacccaaaggctggctcccttaaccccagtgaactttgtctttcagctgatgaccagccgccatcaatcctggaccccccaggttaggagttgggccagttatgggtcaggccctttagcccacgacatccacacagtctgggtttcatccagcccaccccatcctacagaccaggtgcagtttgagaagtgtggcaagagggtggatcggctggatcagcggcgttccaagctgcgcgtggttgggggccatccgggcaactcaccctggacagtcagcttgcggaatcggtgaggcacaactgcctgtctcccacagagaggagctgaggttgtgtcctctgtggttatcccactgggggctgggaatctatccctgcccccagaggtcctagccagaagatggcaggtctagcatctgtcccaggagtctgttacctgtcctaattccccactcctctaggcagggccagcatttctgcggggggtctctagtgaaggagcagtggatactgactgcccggcagtgcttctcctcctggtgagcctcccttgtgtttggggacccagtctcatcccaccttcccccttccccaggcaagctaacaagtgagccttggggcaatggactgagagtcacaaatgacctagcagagcttctctcccagccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagaggtatgtggacaacctgggagggtgtgaggtggggctgggccttgtggcctcagaccctgagtgcccccattcttgctaaagatctgtgaccctgaaccagcgtgtggccctgatctgcctgccccctgaatggtatgtggtgcctccagggaccaagtgtgagattgcaggctggggtgagaccaaaggtaagagcacagtgcacaggactgctggtggccaggaggcccagccctggatcttcctgcaggaccctctccctctccccattcccctcactgcaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgaggttggtggcagggccctgggccagccctggaagggtatggggggctagaaatgaactattttatcatgaagcaggctagtcattgctgtggcccggggccctcatcagttctcctacctgccagggtgactacgggggcccacttgcctgctttacccacaactgctgggtcctggaaggaattataatccccaaccgagtatgcgcaaggtcctgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 31 - RNA, 2621 nt, Homo sapiens
  source 1..2621: mol_type = mRNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 32 - RNA, 2621 nt, Homo sapiens
  source 1..2621: mol_type = mRNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcctgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 33 - DNA, 2621 bp, Homo sapiens
  source 1..2621: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 34 - DNA, 2621 bp, Homo sapiens
  source 1..2621: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcctgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 35 - protein, 725 aa, Homo sapiens
MGLWWVTVQPPARRMGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKHRGRVRESEMCTEGLLAPVGACEGDYGGPLACFTHNCWVLEGIIIPNRVCARSRWPAVFTRVSVFVDWIHKVMRLG

SEQ ID NO: 36 - protein, 725 aa, Homo sapiens [sequence omitted]

SEQ ID NO: 37 - DNA, 5107 bp, Homo sapiens
  source 1..5107: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctggtaagtgcccccaaccctgatccccatctgccttcaggagggggttggccccattctcctattctaggatgagaaaaaagtcggggagccagaggctcagtgggcatggggcagtgaccttgccctcttgagcacagctgggaagccctaggaacacatagacattgcccacttaggcctctattagcacgtctgctctagcactgaagcagtgtcaggaccacacagatgcacgcacacagcaggcagtgacccctcctgagcctgatctacccctctaacctagcatatgcctttgtgcaggtgagagcccagatttggagtctgaatgcctagccagggcccctggctgggtaatgtgatggctctgagccttagcattctcatttgagagatgaggtggggcaagcttcatcacccactgctctcacagagcgtatgtgttagatctgagcccggtgcctgggccactaaacagaggcaccggtgataactaccaagtctgggcctgcttcccaggggaaatttttttcacaagtatctgtgcagggggctagactggcccttgaaagtgcatacagggtccatcccagaagccttgtagctttgatcccctgaatgaacaaagtgtggacatgccaatacacattactgacatgtatgcccacctgacctgcacccactcatgcctactctgcagggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccggtgagtggccactgggcctagataagactgggggcagggaagcctgggctgtggcgttaccctgtgccttcttctctccagggccttccactacaacgtgagcagccatggttgccaactgctgccatggactcaacactcgccccacacgaggctgcggcgttctgggcgctgtgacctcttccagaagaaaggcaagtgggggtggagaggggcagggtgggagacaggggacctcagcccaagttgatcttctgtctcttgctcccagactacgtacggacctgcatcatgaacaatggggttgggtaccggggcaccatggccacgaccgtgggtggcctgccctgccaggcttggagccacaagttcccaaatgatcacaagtgagacaaacaccttccctccgtcccggcctggggccttcccccagcacacactatagtgatgctctgggccctcaggtacacgcccactctccggaatggcctggaagagaacttctgccgtaaccctgatggcgaccccggaggtccttggtgctacacaacagaccctgctgtgcgcttccagagctgcggcatcaaatcctgccgggagggtaagcggcgccgggtcaagctgggagagtggagggacaagcccacgtccatccacgaacccactggctctttgtctccagccgcgtgtgtctggtgcaatggcgaggaataccgcggcgcggtagaccgcacggagtcagggcgcgagtgccagcgctgggatcttcagcacccgcaccagcaccccttcgagccgggcaagtacgcgtaggcggtatcggcgtcctgggggccgggctagggaaggtccaggactccaggggcagggctccgtgtagggcaattgggcggggccagataagccagagtcccagggtcttgttcacgccccattaccgcccccaggttcctcgaccaaggtctggacgacaactattgccggaatcctgacggctccgagcggccatggtgctacactacggatccgcagatcgagcgagagttctgtgacctcccccgctgcggtaggcggcggggaccaggcctgggagggtacctgggaaccttggggaggggcgtggcttggccggggaggtaagaggggctgggcgtgacctgagagcataccccgtggagtaccgtacacctgggaaaggcgggtttggtcccagccccagagggatctcagctctcgctcggggcccgacctatctcggtccatctaagggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaagtgaggtggggggggggggcgggcgttgggacgtgctgctgcgggtgagacgggaggaaggtagtcacgggctcaaggctggaggctggcgggctagggctgagtggagcgcctgcttagagaccttcgggagaacttctgccggaaccccgacggctcagaggcgccctggtgcttcacactgcggcccggcatgcgcgcggccttttgctaccagatccggcgttgtacagacgacgtgcggccccagggtgaggcccaagcttgggggctacagagccgggctggaagcctggaaccggagggccggggcgaggtctcggcctgatggctgcccgcacccgccacagactgctaccacggcgcaggggagcagtaccgcggcacggtcagcaagacccgcaagggtgtccagtgccagcgctggtccgctgagacgccgcacaagccgcagtgagtccctggtgctcccggccccgccagggccctaaccctggggcggcatgctttggtgtctgggaccagagcctggaaatggttgagactaccctgccacgattttgctcccgcttccgcctaggttcacgtttacctccgaaccgcatgcacaactggaggagaacttctgccggaacccagatggggatagccatgggccctggtgctacacgatggacccaaggaccccattcgactactgtgccctgcgacgctgcggtgagcactagtgacgcttcccccatgaccctgcctcagcccccacccaaaggctggctcccttaaccccagtgaactttgtctttcagctgatgaccagccgccatcaatcctggaccccccaggttaggagttgggccagttatgggtcaggccctttagcccacgacatccacacagtctgggtttcatccagcccaccccatcctacagaccaggtgcagtttgagaagtgtggcaagagggtggatcggctggatcagcggcgttccaagctgcgcgtggttgggggccatccgggcaactcaccctggacagtcagcttgcggaatcggtgaggcacaactgcctgtctcccacagagaggagctgaggttgtgtcctctgtggttatcccactgggggctgggaatctatccctgcccccagaggtcctagccagaagatggcaggtctagcatctgtcccaggagtctgttacctgtcctaattccccactcctctaggcagggccagcatttctgcggggggtctctagtgaaggagcagtggatactgactgcccggcagtgcttctcctcctggtgagcctcccttgtgtttggggacccagtctcatcccaccttcccccttccccaggcaagctaacaagtgagccttggggcaatggactgagagtcacaaatgacctagcagagcttctctcccagccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagaggtatgtggacaacctgggagggtgtgaggtggggctgggccttgtggcctcagaccctgagtgcccccattcttgctaaagatctgtgaccctgaaccagcgtgtggccctgatctgcctgccccctgaatggtatgtggtgcctccagggaccaagtgtgagattgcaggctggggtgagaccaaaggtaagagcacagtgcacaggactgctggtggccaggaggcccagccctggatcttcctgcaggaccctctccctctccccattcccctcactgcaggtacgggtaatgacacagtcctaaatgtggccttgctgaatgtcatctccaaccaggagtgtaacatcaagcactgaggacgtgtgcgggagagtgagatgtgcactgagggactgttggcccctgtgggggcctgtgaggttggtggcagggccctgggccagccctggaagggtatggggggctagaaatgaactattttatcatgaagcaggctagtcattgctgtggcccggggccctcatcagttctcctacctgccagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 38 - RNA, 2621 nt, Homo sapiens
  source 1..2621: mol_type = mRNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcactgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 39 - DNA, 2621 bp, Homo sapiens
  source 1..2621: mol_type = genomic DNA
gaagctggggcaagtaattttccccaatttacagggaaaaaccgaaattcagaaaagtttaatgtcacccaggggctggagcccagacctctggcagctctcactttcacaatgcccttgggctgactaggctgcagaggggtttcaccccaaccccagggcacctcaagtgtccccaccaaaccttcctaacacctgtccactaagctgtactaggcccttgcaactgacctatgggacctgaggcctggcccctcatggctcctgtcaccaggtctcaggtcagggtccagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcactgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgta

SEQ ID NO: 40 - protein, 650 aa, Homo sapiens
MGLWWVTVQPPARRMGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKH